(19) United States
(12) Patent Application Publication
(10) Pub. No.: US 2015/0031686 A1
(43) Pub. Date: Jan. 29, 2015

(54) PHENYL-OXAZOLYL DERIVATIVES, PREPARATION METHOD THEREOF, AND RELATED APPLICATION OF THE PHENYL-OXAZOLYL DERIVATIVES AS AN IMPDH INHIBITOR

(71) Applicant: INSTITUTE OF MEDICINAL BIOTECHNOLOGY, CHINESE ACADEMY OF MEDICAL SCIENCES, Beijing (CN)

(72) Inventors: Zhaojin Zhong, Beijing (CN); Zhuorong Li, Beijing (CN); Dajun Zhang, Beijing (CN); Yuhuan Li, Beijing (CN); Zhen Wang, Beijing (CN); Rongmei Gao, Beijing (CN); Jiajia Shen, Beijing (CN)

(21) Appl. No.: 14/383,616

(22) PCT Filed: Mar. 6, 2013
(86) PCT No.: PCT/CN2013/000234

§ 371 Date: Sep. 8, 2014

(30) Foreign Application Priority Data
Mar. 8, 2012 (CN) CN201210060269.X

(51) Int. Cl. C07D 263/32; C07D 417/12; C07D 413/12

(52) U.S. Cl. CPC C07D 263/32; C07D 413/12; C07D 417/12
USPC 514/225.2; 548/235; 514/374; 548/236; 548/156; 514/367; 546/209; 514/326; 544/238; 514/252.05; 544/333; 514/256; 544/405; 514/255.05; 546/176; 514/314; 544/235; 514/248; 544/38

(57) ABSTRACT

Disclosed are phenyl-oxazolyl derivatives having a general formula (I), a preparation method thereof, and an application of the phenyl-oxazolyl derivatives as an inosine monophosphate dehydrogenase (IMPDH) inhibitor.

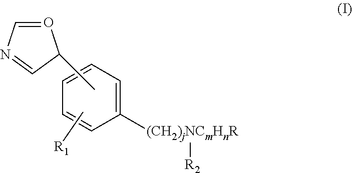

### PRIORITY STATEMENT

The present application requires the priority of the Chinese temporary patent application applied on Mar. 8, 2012, application number 201210060269.X, title of the invention “Phenyl-oxazolyl derivatives, preparation methods thereof, and related application of the phenyl-oxazolyl derivatives as an impdh inhibition”, the specification is incorporated by reference herein in its entirety.

### TECHNICAL FIELD

The present invention related to a group of phenyl-oxazolyl derivatives, and the preparation methods thereof, it also related to the relevant applications of phenyl-oxazolyl derivatives as inosine monophosphate dehydrogenase (IMPDH) inhibitors. This invention belongs to the biomedical field.

### BACKGROUND OF THE INVENTION

Inosine monophosphate dehydrogenase (IMPDH) is a key enzyme in purine nucleotide biosynthesis, depending on nicotinamide adenine dinucleotide (NAD), and it catalyzes the speed limiting step of de novo catalytic synthesis of guanine nucleotide oxidating inosinic acid (IMP) into inosine monophosphate (XMP). XMP is then converted to GMP under the effect of GMP synthetase. These reactions are essential for cell growth and proliferation. Apart from a few protozoan parasites, IMPDH GMP is present in nearly every organism. IMPDH inhibition will lead to inhibition of proliferation and many important cellular responses. Human IMPDH are divided into two types, type I (hIMPDH1) and Type II (hIMPDH2), they have 84% homology. Studies have shown that, hIMPDH2 is selectively upregulated in proliferating cells, is significantly expressed in the activated peripheral lymphocytes; in most quiescent cells, hIMPDH1 expression is dominant. hIMPDH1 also been shown to play a critical role in angiogenesis. Therefore, IMPDHs become important targets in the discovery of anti-virus, anti-cancer, drugs of immune suppression and other kinds.

Based on their binding activity center points, IMPDH inhibitors can be divided into IMP- and NAD-point categories. All the inhibitory mechanism is by blocking the IMP and NAD or by changing their point conformations, thereby possibly interfering or even terminating substrate activity process.

Reversible and irreversible IMP point inhibitors include a number of compounds and their monophosphates, such as ribavirin (Hager P W, Biochem Pharmacol 1995; 49 (9): 1323-1329), 3-deazaguanosine (Cook P D, J Am Chem Soc 1976, 98 (6): 1492-1498.; Streeter D G, Biochem Pharmacol 1976, 25 (21): 2413-2415), mizoribine (Hager P W, Biochem Pharmacol 1995; 49 (9): 1323-132; Kerr K M, Biochemistry 1997, 36 (43): 13365-13373), 6-chloropurine riboside (Antonino L C, Biochemistry 1994., 33 (7): 1760-1765), and 2-vinyl inosine (Pal S, Bioorg Med Chem 2002, 10 (11): 3615-3618; Nair V, Clin Microbiol Rev 2001, 14 (2): 382-397) and the like. Ribavirin has been used in the treatment of respiratory syncytial virus infection, as well as, in combination with α-interferon, in the treatment of hepatitis C. Mizoribine has been approved for the treatment of organ transplant rejection, rheumatoid arthritis, primary nephrotic syndrome, lupus nephritis, dermatomyositis and autoimmune skin diseases. X-ray crystal structure of the complexes formed between IMPDH and 6-chloropurine nucleoside phosphate, or 2-ethylene inosine phosphate, all indicated formation of covalent bonds between the inhibitors and the enzyme Cys331.

The inhibitors with the point NAD as binding target, such as tiazofurin (De Clercq E. Clin Microbiol Rev 2001, 14 (2): 382-397; Minakawa N, Curr Med Chem 1999; 6 (7): 615-628), are non-competitive IMPDH inhibitor of NAD⁺, they have anti-proliferative and anti-viral activity, and its selenium analogues displayed in vivo anti-tumor activity. Selenazole-nucleoside (Jayaram H N, Biochem Pharmacol 1983; 32 (17): 2633-2636; Streeter D G, Biocheml Biophys Res Commun 1983, 115 (2): 544-550.) showed in vitro anti-DNA and RNA viral activity. Through the active metabolite of benzamide riboside, amide adenine dinucleotide, it showed inhibitory activity competing with NAD⁺ in IMPDH catalytic process.

Mycophenolic acid (MPA) is suitable for binding on the nicotinamide side of NAD binding point without having to be activated. Based on the drug design with structural consideration on MPA and other binding modes, a series of IMPDH inhibitors, such as compound VX-497 (Merimepodib), VX-148 and AVN944 etc. (Lizbeth Hedstrom. Chem. Rev. 2009, 109, 2903-2928) binding on sites of urea type, have been obtained, and by further structural modifications, it produced IMPDH inhibitors containing structures of oxazole-indole, cyanoindole, pyridinyl-indole, isoquinoline or acridone, and the like.

MPA can induce necrosis of lymphocytes and inhibit tumor cell growth. A series of mycophenolic acid adenine nucleotides showed a strong anti-leukemic effects as well as the ability to induce cell differentiation, is thus promising drug candidates for treatment of chronic myeloid leukemia (Sonja B Braun-Sand, Future Med. Chem. 2010, 2 (1): 81-92; Rejman D, J Med Chem, 2006, 49 (16): 5018-5022). SAHA (Suberoylanilide hydroxamic acid) can simultaneously inhibit IMPDH and HDAC, and can be used for the treatment of cutaneous T-cell lymphoma (Liqiang Chen, J Med Chem, 2007, 50, 6685-6691). AVN944, a specific noncompetitive inhibitor of IMPDH, is expected to become a promising drug for the treatment of prostate cancer. In combination with gemcitabine, AVN944 has entered clinical trials for treatment of pancreatic cancer (Floryk D, Int J Cancer, 2008, 123 (10): 2294-2302.).

VX-497 is an anti-virus candidate compound, in in vitro experiments against hepatitis B virus, human cytomegalovirus, respiratory syncytial virus, herpes simplex type 1 virus, encephalomyocarditis virus and Venezuelan equine encephalomyelitis virus, VX-497 demonstrated stronger antiviral activity than ribavirin. In experiments tested in combination with interferon, VX-497 also demonstrated better results than those from combined usage of ribavirin and interferon, its therapeutic effect on hepatitis C has entered phase H clinical trials (Markland W, Antimicrob Agents Chemother 2000, 44, (4) 859-866).

IMPDH inhibitors are also used in immune suppression, antimicrobial and antiparasitic researches. As prodrug of MPA, mycophenolate mofetil has been applied to solid organ transplantation. VX-497, VX-148 and other drugs were used in immunosuppressive therapeutical researches, they are promising as ideal candidate drugs.

In drug discovery researches using IMPDHs as a targets, the present invention demonstrates that, a series of new phenyl-oxazolyl derivatives showing potent IMPDH inhibitory activity, as well as anti-virus (COX-B3), anti-tumor (Hepatoma cells, human lung adenocarcinoma cells) effects and immune inhibition activity. So far, no report on compounds of the invention and their effects has been seen from literatures in China and abroad. Development of new drugs for clinical applications using IMPDH as target will provide more effective new anti-viral, anti-cancer and immune suppression drugs.

### SUMMARY OF THE INVENTION

The purpose of the present invention is to provide a set of phenyl-oxazolyl derivatives and their preparation methods, as well as their related application as an inosine monophosphate dehydrogenase (IMPDH) inhibitor.

To achieve the above objects, the present invention adopts the following technical schemes:

A group of phenyl-oxazolyl derivatives or their pharmaceutically acceptable salts thereof, having the general structure shown in (I):

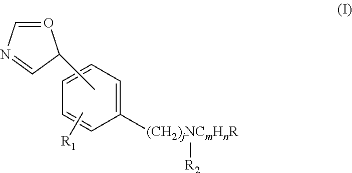

Wherein:

R₁ represents one of the following: H, a halogen, hydroxyl, C₁-C₃ alkyl, C₁-C₃ alkoxyl;

R₂ represents one of the following: H, a substituted or unsubstituted, saturated or unsaturated C₁-C₁₂ alkyl, a carbonyl or a sulfonyl, or absent;

R represents one of the following: H, cyano group, substituted or unsubstituted, saturated or unsaturated C₁-C₁₂ alkyl, C₁-C₁₂ alkoxyl or aryloxyl, C₁-C₁₂ alkylmercapto or arylmercapto group, amino group, substituted amino group, sulfonic acid group, sulfonyl, substituted or unsubstituted monocyclic to tricyclic aryl group, substituted or unsubstituted heterocyclic group;

Said heterocyclic group is selected from one of the following: a five to six membered monocyclic heterocycles, five to six membered bicyclic or tricyclic heterocycles;

wherein the heterocyclic group containing 1 to 3 hetero atoms;

wherein the heteroatoms are N, O, S;

j=0-1, m=0-3, n=0-6, j, in and n may be the same or different;

wherein said five membered monocyclic heterocycle is selected from one of the following: substituted or unsubstituted thienyl, furyl, pyrrolyl, isoxazolyl, thiazolyl, imidazolyl, pyrazolyl or triazolyl group;

wherein said six membered monocyclic heterocycle is selected from one of the following: substituted or unsubstituted piperidinyl, pyridyl, pyranyl, pyridazinyl, pyrimidinyl or pyrazinyl group;

wherein said bicyclic heterocycle is selected from one of the following: substituted or unsubstituted indolyl, benzothienyl, benzothiazolyl, benzoxazolyl, benzopyranyl, thiobenzopyranyl, quinolyl, cinnolinyl, indazolyl, benzooxadiazolyl or benzothiadiazolyl;

wherein said tricyclic heterocycle is selected from one of the following: substituted or unsubstituted dibenzofuranyl, dibenzothiophene group, acridinyl, or phenothiazinyl.

The methods to prepare general formula (I) compounds are according to the following equation:

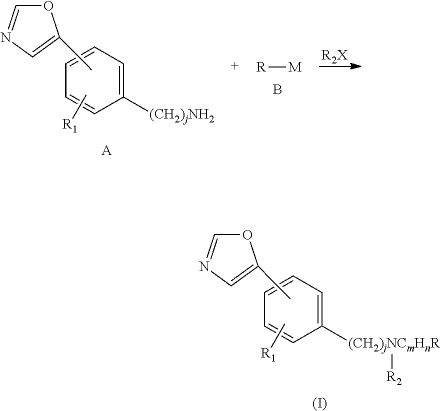

Compound A and B are mixed and dissolved in a solvent, after finishing the reaction, the reactant is further reacted with R₂X to obtain general formula (I) compounds;

wherein, R₁, R₂, R, j, m, n are as defined above,

M, X represents an formyl, a halogen or an acyl.

A pharmaceutically acceptable salt of the phenyl-oxazolyl derivatives can be obtained by salifying the general formula (I) compound with a corresponding acid, wherein the acid is selected from inorganic or organic acids. Preferably, the inorganic acid is selected from hydrochloric acid, hydrobromic acid or sulfuric acid; the organic acid is selected acetic acid, trifluoroacetic acid, lactic acid, succinic acid, fumaric acid, maleic acid, citric acid, benzoic acid, methanesulfonic acid or p-toluene sulfonic acid.

A pharmaceutical composition, comprising a therapeutically effective amount of a Formula (I) compound or a pharmaceutically acceptable salt thereof as an active ingredient, in association with one or more pharmaceutically acceptable carrier components.

The applications of phenyl-oxazolyl derivatives or pharmaceutically acceptable salts thereof in the manufacture of a drugs as an inhibitor of IMPDH, the said drugs include antiviral drugs, anticancer drugs and immunosuppressive drugs.

The application of pharmaceutical compositions in the manufacture of IMPDH inhibiting drugs, the said drugs include antiviral drugs, anticancer drugs and immunosuppressive drugs.

Adopting the above technical schemes, the present invention has the following advantages:

Through researches on the design, synthesis and structure-activity relationships of phenyl-oxazolyl derivatives of the general formula (I), the present invention obtained a new class of IMPDH inhibitor, laying the foundation for the development and application of drugs and their pharmaceutical compositions with functions relating to anti-viral, anti-tumor, and immune suppression effects on the basis of these compounds.

### EMBODIMENTS

The starting material and the desired product for the synthesis of phenyl-oxazolyl derivatives and pharmaceutically acceptable salt thereof in the present invention can be determined according to the R, R₁ and R₂ in the structural design, for example, when R₂ is absent, the target product is N-(thien-2-yl methylene) 3-methoxy-4-(oxazol-5-yl) aniline, the specific reaction steps are as follows:

Dissolve 3-methoxy-4-(oxazol-5-yl) aniline (Compound A) in absolute ethanol, add in 2-thiophene aldehyde (Compound B), stir at room temperature until starting material 3-methoxy-4-(oxazol-5-yl) aniline disappears to obtain a Schiff base, which is then isolated and purified by using crystallization or chromatography.

In the case of presence of R₂, following target product of N-methyl-N-(5-methyl-thien-3-yl methyl) 3-methoxy-4-(oxazol-5-yl) aniline is used as an example, the specific steps are as follows:

Mix 3-methoxy-4-(oxazol-5-yl) aniline (A) with 5-methylthiophene-3-carbaldehyde (B) and dissolve the mixture in ethanol, stir at room temperature until the starting materials completely dissolves to obtain Schiff base as the intermediate product, then lower the temperature of the reaction solution to 0-10° C., add in a reducing agent (such as NaBH₄) slowly, until the complete dissolution of the Schiff base, isolate the product and dissolve it in acetone, stir in the presence of anhydrous K₂CO₃, add methyl iodide (compound R₂X), until the raw material completely dissolves, isolate the product and purify it by using recrystallization or chromatography.

Above two synthetic reaction procedures are represented by the following equations:

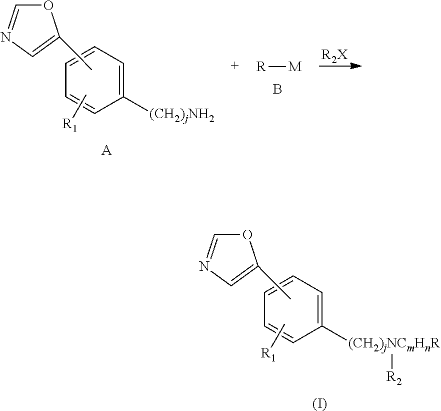

wherein M, X, R, R₁, R₂, and j, in, and n are as defined above.

In the above processes, the starting material 3-methoxy-4-(oxazol-5-yl) aniline, 2-thiophenealdehyde, 5-methyl-thiene-3-yl aldehyde, etc. can be purchased or can be prepared by known methods.

The present invention selects the compounds defined as phenyl-oxazolyl derivatives from a large number of candidate compounds, the structure examples of the invented compounds are listed, but not limited in Table 1.

TABLE 1
A part of the structures of compounds included in this invention

| | | | Molecular | |
|---|---|---|---|---|
| | Chemical name | Structure | formulae | MW |
| 1 | N-(thien-2-ylmethyl)-3- methoxy-4-(oxazol-5-yl) aniline | 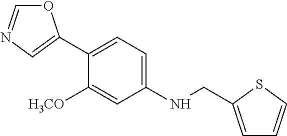 [SMILES: COc1cc(NCc2cccs2)ccc1-c1cnco1] | C₁₅H₁₄N₂O₂S | 286.35 |
| 2 | N-(5-methylthien-2-ylmethyl)- 3-methoxy-4-(oxazol-5-yl) aniline | 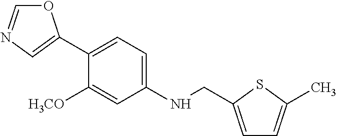 [SMILES: COc1cc(NCc2ccc(C)s2)ccc1-c1cnco1] | C₁₆H₁₆N₂O₂S | 300.38 |
| 3 | N-(5-ethylthien-2-ylmethyl)- 3-methoxy-4-(oxazol-5-yl) aniline | 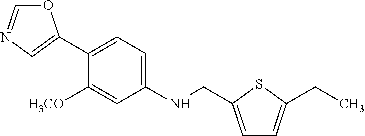 [SMILES: CCc1ccc(CNc2ccc(-c3cnco3)c(OC)c2)s1] | C₁₇H₁₈N₂O₂S | 314.40 |
| 4 | N-(5-chlorothien-2-ylmethyl)- 3-methoxy-4-(oxazol-5-yl) aniline | 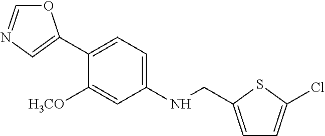 [SMILES: COc1cc(NCc2ccc(Cl)s2)ccc1-c1cnco1] | C₁₅H₁₃ClN₂O₂S | 320.79 |
| 5 | N-(5-bromothien-2-ylmethyl)- 3-methoxy-4-(oxazol-5-yl) aniline | 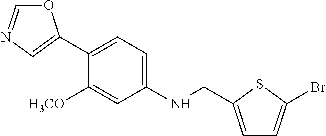 [SMILES: COc1cc(NCc2ccc(Br)s2)ccc1-c1cnco1] | C₁₅H₁₃BrN₂O₂S | 365.24 |
| 6 | N-(5-phenylthien-2-ylmethyl)- 3-methoxy-4-(oxazol-5-yl) aniline | 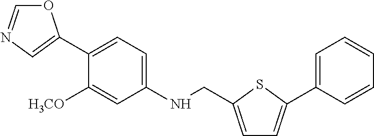 [SMILES: COc1cc(NCc2ccc(-c3ccccc3)s2)ccc1-c1cnco1] | C₂₁H₁₈N₂O₂S | 362.45 |
| 7 | 5-(3-methoxy-4-oxazol-5-yl- anilinomethyl)-thien-2-yl carboxylic acid | 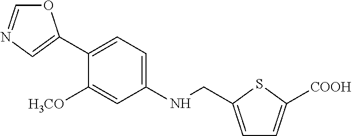 [SMILES: COc1cc(NCc2ccc(C(=O)O)s2)ccc1-c1cnco1] | C₁₆H₁₄N₂O₄S | 330.36 |
| 8 | N-(benzothiazol-2-ylmethyl)- 3-methoxy-4-(oxazol-5-yl) aniline | 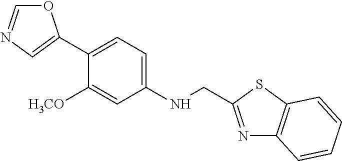 [SMILES: COc1cc(NCc2nc3ccccc3s2)ccc1-c1cnco1] | C₁₈H₁₅N₃O₂S | 337.40 |
| 9 | N-(thien-2-yl methylene)-3- methoxy-4-(oxazol-5-yl) aniline | 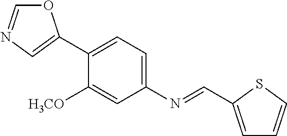 [SMILES: COc1cc(/N=C/c2cccs2)ccc1-c1cnco1] | C₁₅H₁₂N₂O₃S | 284.33 |
| 10 | N-(benzothien-2-ylmethyl)- 3-methoxy-4-(oxazol-5-yl) aniline | 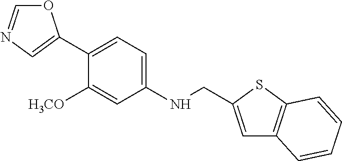 [SMILES: COc1cc(NCc2cc3ccccc3s2)ccc1-c1cnco1] | C₁₉H₁₆N₂O₂S | 336.41 |
| 11 | N-(thien-3-yl methyl)-3- methoxy-4-(oxazol-5-yl) aniline | 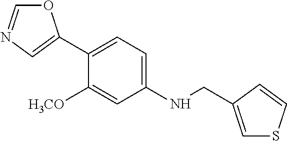 [SMILES: COc1cc(NCc2ccsc2)ccc1-c1cnco1] | C₁₅H₁₄N₂O₂S | 286.35 |
| 12 | N-methyl-N-(5-methylthien-3-yl methyl)-3-methoxy-4-(oxazol-5-yl) aniline | 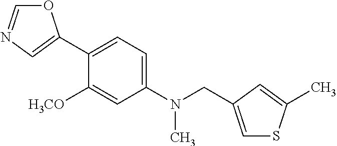 [SMILES: COc1cc(N(C)Cc2csc(C)c2)ccc1-c1cnco1] | C₁₇H₁₈N₂O₂S | 314.40 |
| 13 | N-(5-ethyl-2-methylthien-3- ylmethyl)-N-(3-methoxy-4-oxazole- 5-phenyl) propyn-2-ylamine | 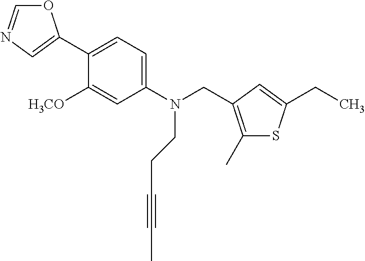 [SMILES: CC#CCCN(Cc1cc(CC)sc1C)c1ccc(-c2cnco2)c(OC)c1] | C₂₁H₂₂N₂O₂S | 366.48 |
| 14 | N-(5-chlorothien-3-ylmethyl)- 3-methoxy-4-(oxazol-5-yl) aniline | 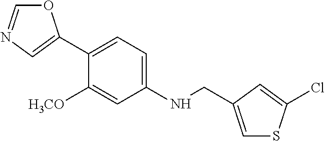 [SMILES: COc1cc(NCc2csc(Cl)c2)ccc1-c1cnco1] | C₁₅H₁₃ClN₂O₂S | 320.79 |
| 15 | N-(5-bromothien-3-ylmethyl)- 3-methoxy-4-(oxazol-5-yl) aniline | 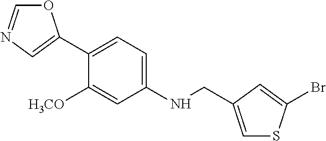 [SMILES: COc1cc(NCc2csc(Br)c2)ccc1-c1cnco1] | C₁₅H₁₃BrN₂O₂S | 365.24 |
| 16 | N-(5-phenylthien-3-ylmethyl)- 3-methoxy-4-(oxazol-5-yl) aniline | 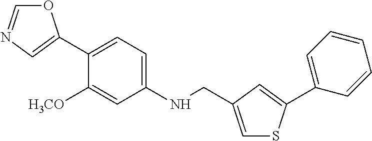 [SMILES: COc1cc(NCc2csc(-c3ccccc3)c2)ccc1-c1cnco1] | C₂₁H₁₈N₂O₂S | 362.45 |
| 17 | N-(1-methyl-1H-imidazol-5- ylmethyl)-3-methoxy-4-(oxazol- 5-yl) aniline | 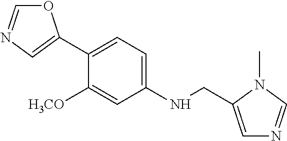 [SMILES: COc1cc(NCc2cncn2C)ccc1-c1cnco1] | C₁₅H₁₆N₄O₂ | 284.32 |
| 18 | N-(5-methoxy-thien-3-yl methyl)- 3-methoxy-4(oxazol-5-yl) aniline | 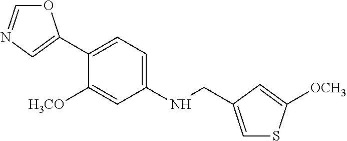 [SMILES: COc1cc(CNc2ccc(-c3cnco3)c(OC)c2)cs1] | C₁₆H₁₆N₂O₃S | 316.37 |
| 19 | N-(5-methylthien-3-ylmethylene)- 3-methoxy-4-(oxazol-5-yl) aniline | 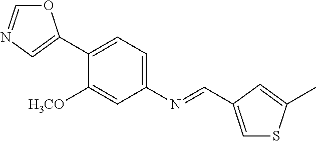 [SMILES: COc1cc(/N=C/c2csc(C)c2)ccc1-c1cnco1] | C₁₆H₁₄N₂O₂S | 298.36 |
| 20 | N-(benzothien-3-ylmethyl)- 3-methoxy-4-(oxazol-5-yl) aniline | 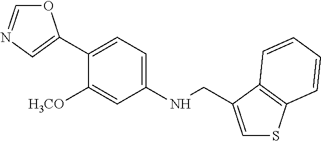 [SMILES: COc1cc(NCc2csc3ccccc23)ccc1-c1cnco1] | C₁₉H₁₆N₂O₂S | 336.41 |
| 21 | N-(thien-2-ylmethyl)-2- methoxy-5-(oxazol-5-yl) aniline | 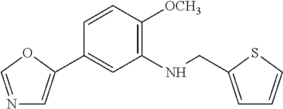 [SMILES: COc1ccc(-c2cnco2)cc1NCc1cccs1] | C₁₅H₁₄N₂O₂S | 286.35 |
| 22 | N-(5-methylthien-2-ylmethyl)- 2-methoxy-5-(oxazol-5-yl) aniline | 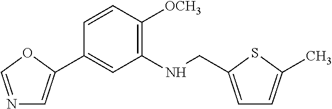 [SMILES: COc1ccc(-c2cnco2)cc1NCc1ccc(C)s1] | C₁₆H₁₆N₂O₂S | 300.38 |
| 23 | N-(3,5-dimethylisoxazol-4- ylmethyl)-2-methoxy-5-(oxazol-5- yl) aniline | 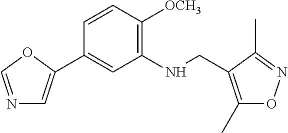 [SMILES: COc1ccc(-c2cnco2)cc1NCc1c(C)noc1C] | C₁₆H₁₇N₃O₃ | 299.33 |
| 24 | N-(5-chloromethylthien-2- ylmethyl)-2-methoxy-5-(oxazol-5- yl) aniline | 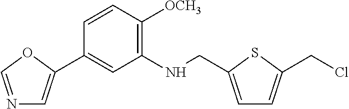 [SMILES: COc1ccc(-c2cnco2)cc1NCc1ccc(CCl)s1] | C₁₆H₁₅ClN₂O₂S | 334.82 |
| 25 | N-(5-bromomethylthien-2- ylmethyl)-2-methoxy-5-(oxazol-5- yl) aniline | 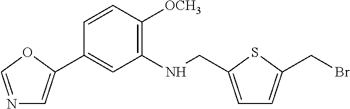 [SMILES: COc1ccc(-c2cnco2)cc1NCc1ccc(CBr)s1] | C₁₆H₁₅BrN₂O₂S | 379.27 |
| 26 | N-(5-phenylthien-2-ylmethyl)- 2-methoxy-5-(oxazol-5-yl) aniline | 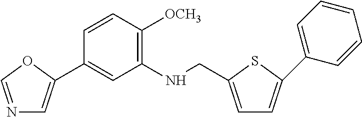 [SMILES: COc1ccc(-c2cnco2)cc1NCc1ccc(-c2ccccc2)s1] | C₂₁H₁₈N₂O₂S | 362.45 |
| 27 | N-(5-chloro-1,3-dimethyl-1H- pyrazol-4-ylmethyl)-2-methoxy- 5-(oxazol-5-yl) aniline | 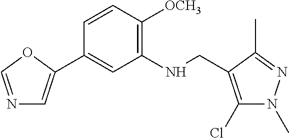 [SMILES: COc1ccc(-c2cnco2)cc1NCc1c(C)nn(C)c1Cl] | C₁₆H₁₇ClN₄O₂ | 332.79 |
| 28 | N-(2-phenyl-2H-1,2,3-triazol- 4-ylmethyl)-(2-methoxy-5-oxazole- 5-yl) aniline | 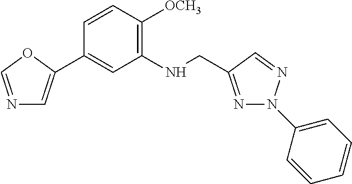 [SMILES: COc1ccc(-c2cnco2)cc1NCc1cnn(-c2ccccc2)n1] | C₁₉H₁₇N₅O₂ | 347.38 |
| 29 | N-(4-chlorothien-2-ylmethylene)- 2-methoxy-5-(oxazol-5-yl)- aniline | 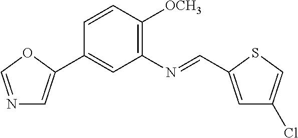 [SMILES: COc1ccc(-c2cnco2)cc1/N=C/c1cc(Cl)cs1] | C₁₅H₁₁ClN₂O₂S | 318.78 |
| 30 | N-(5-chlorobenzothien-2-ylmethyl)- 2-methoxy-5-(oxazol-5-yl) aniline | 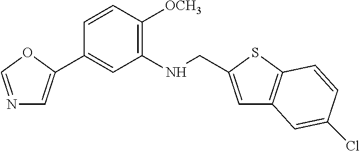 [SMILES: COc1ccc(-c2cnco2)cc1NCc1cc2cc(Cl)ccc2s1] | C₁₉H₁₅ClN₂O₂S | 370.85 |
| 31 | N-(thien-3-yl methyl)-2-methoxy- 5-(oxazol-5-yl) aniline | 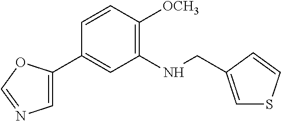 [SMILES: COc1ccc(-c2cnco2)cc1NCc1ccsc1] | C₁₅H₁₄N₂O₂S | 286.35 |
| 32 | N-(5-methylthien-3-yl methyl)- N-(2-methoxy-5-oxazol-5-yl) acrylamide | 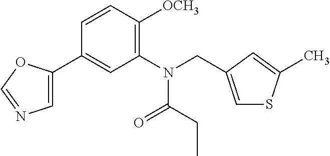 [SMILES: CCC(=O)N(Cc1csc(C)c1)c1cc(-c2cnco2)ccc1OC] | C₁₉H₂₀N₂O₃S | 356.44 |
| 33 | N-(2-methoxy-5-oxazol-5-yl phenyl)-1-methylpiperidin-4-yl methylamine | 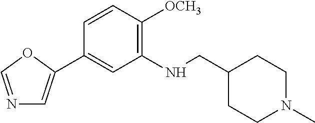 [SMILES: COc1ccc(-c2cnco2)cc1NCC1CCN(C)CC1] | C₁₇H₂₃N₃O₂ | 301.39 |
| 34 | N-(5-chlorothien-3-ylmethyl)- 2-methoxy-5-(oxazol-5-yl) aniline | 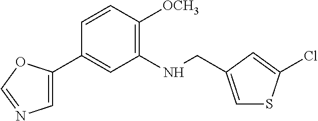 [SMILES: COc1ccc(-c2cnco2)cc1NCc1csc(Cl)c1] | C₁₅H₁₃ClN₂O₂S | 320.79 |
| 35 | N-(5-bromothien-3-ylmethyl)- 2-methoxy-5-(oxazol-5-yl) aniline | 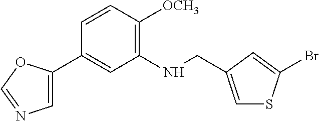 [SMILES: COc1ccc(-c2cnco2)cc1NCc1csc(Br)c1] | C₁₅H₁₃BrN₂O₂S | 365.24 |
| 36 | N-(5-phenylthien-3-ylmethyl)- 2-methoxy-5-(oxazol-5-yl) aniline | 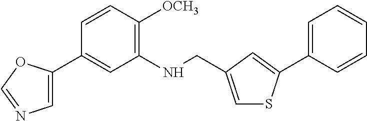 [SMILES: COc1ccc(-c2cnco2)cc1NCc1csc(-c2ccccc2)c1] | C₂₁H₁₈N₂O₂S | 362.45 |
| 37 | N-(tetrahydropyran-4-ylmethyl)- 2-methoxy-5-oxazol-5-yl aniline | 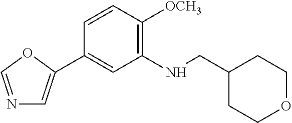 [SMILES: COc1ccc(-c2cnco2)cc1NCC1CCOCC1] | C₁₆H₂₀N₂O₃ | 288.35 |
| 38 | N-(pyridazin-3-yl-methyl)-2- methoxy-5-oxazol-5-yl aniline | 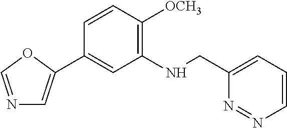 [SMILES: COc1ccc(-c2cnco2)cc1NCc1cccnn1] | C₁₅H₁₄N₄O₂ | 282.30 |
| 39 | N-(thien-3-ylmethylene)-2- methoxy-5-(oxazol-5-yl)-aniline | 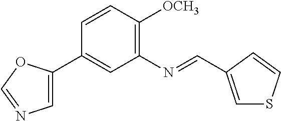 [SMILES: COc1ccc(-c2cnco2)cc1/N=C/c1ccsc1] | C₁₅H₁₂N₂O₃S | 284.33 |
| 40 | N-(6-bromo-7-methyl-benzothien- 3-ylmethyl)-2-methoxy-5- oxazol-5-yl aniline | 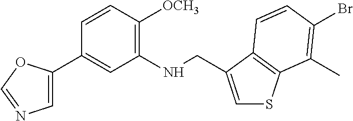 [SMILES: COc1ccc(-c2cnco2)cc1NCc1csc2c(C)c(Br)ccc12] | C₂₀H₁₇BrN₂O₂S | 429.33 |
| 41 | N-(furan-2-yl methyl)-3- methoxy-4-(oxazol-5-yl) aniline | 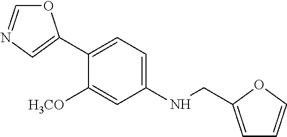 [SMILES: COc1cc(NCc2ccco2)ccc1-c1cnco1] | C₁₅H₁₄N₂O₃ | 270.29 |
| 42 | N-(5-methylfuran-2-ylmethyl)- 3-methoxy-4-(oxazol-5-yl) aniline | 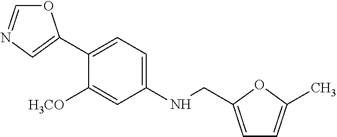 [SMILES: COc1cc(NCc2ccc(C)o2)ccc1-c1cnco1] | C₁₆H₁₆N₂O₃ | 284.31 |
| 43 | N-(2,4,6-trichloropyrimidin-5- ylmethyl)-3-methoxy-4-(oxazol- 5-yl) aniline | 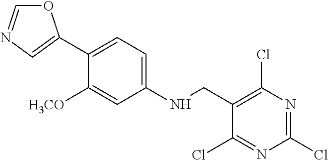 [SMILES: COc1cc(NCc2c(Cl)nc(Cl)nc2Cl)ccc1-c1cnco1] | C₁₅H₁₁Cl₃N₄O₂ | 385.64 |
| 44 | N-(3,5-dichloroperazin-2-ylmethyl)- 3-methoxy-4-(oxazol-5- yl) aniline | 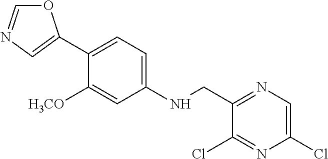 [SMILES: COc1cc(NCc2ncc(Cl)nc2Cl)ccc1-c1cnco1] | C₁₅H₁₂Cl₂N₄O₂ | 351.19 |
| 45 | N-(5-bromofuran-2-ylmethyl)- 2-methoxy-5-(oxazol-5-yl) aniline | 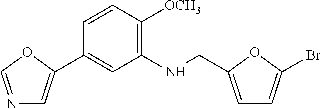 [SMILES: COc1ccc(-c2cnco2)cc1NCc1ccc(Br)o1] | C₁₅H₁₃BrN₂O₃ | 349.18 |
| 46 | N-[5-(3-chlorophenyl)-furan-2- ylmethyl]-2-methoxy-5-(oxazol- 5-yl) aniline | 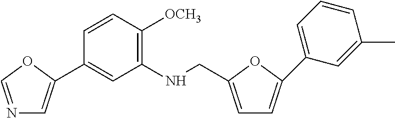 [SMILES: COc1ccc(-c2cnco2)cc1NCc1ccc(-c2cccc(C)c2)o1] | C₁₅H₁₃BrN₂O₃ | 380.83 |
| 47 | N-2-(3,4,5- trimethoxyphenylaminocarbonyl) furan-5-ylmethyl 3-methoxy-4-(oxazol-5-yl)aniline | 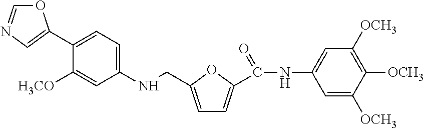 [SMILES: COc1cc(NCc2ccc(C(=O)Nc3cc(OC)c(OC)c(OC)c3)o2)ccc1-c1cnco1] | C₂₁H₁₇ClN₂O₃ | 479.49 |
| 48 | N-(5-methoxymethylfuran-2- ylmethyl)-2-methoxy-5-(oxazol- 5-yl) aniline | 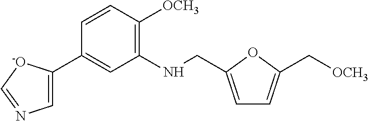 | C₁₇H₁₈N₂O₄ | 314.24 |
| 49 | N-(5-nitrofuran-2-methylidene)- 3-methoxy-4-(oxazol-5-yl) aniline | 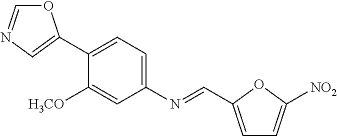 [SMILES: COc1cc(/N=C/c2ccc([N+](=O)[O-])o2)ccc1-c1cnco1] | C₁₅H₁₁N₃O₅ | 313.27 |
| 50 | N-(benzofuran-3-ylmethyl)-2- methoxy-5-(oxazol-5-yl) aniline | 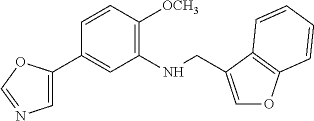 [SMILES: COc1ccc(-c2cnco2)cc1NCc1coc2ccccc12] | C₁₉H₁₆N₂O₃ | 320.35 |
| 51 | N-(1-methyl-1H-pyrrol-2-ylmethyl)- 3-methoxy-4-(oxazol-5- yl) aniline | 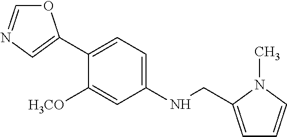 [SMILES: COc1cc(NCc2cccn2C)ccc1-c1cnco1] | C₁₆H₁₇N₃O₂ | 283.33 |
| 52 | N-(5-methoxy-1H-indol-3- ylmethyl)-3-methoxy-4-(oxazol-5- yl) aniline | 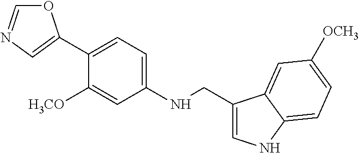 [SMILES: COc1ccc2[nH]cc(CNc3ccc(-c4cnco4)c(OC)c3)c2c1] | C₂₀H₁₉N₃O₃ | 349.39 |
| 53 | N-(1H-indol-3-yl methyl)-3- methoxy-4-(oxazol-5-yl) aniline | 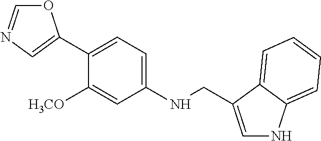 [SMILES: COc1cc(NCc2c[nH]c3ccccc23)ccc1-c1cnco1] | C₁₉H₁₇N₃O₂ | 319.36 |
| 54 | N-(4-methylthiazol-5-yl methyl)- 3-methoxy-4-(oxazol-5-yl) aniline | 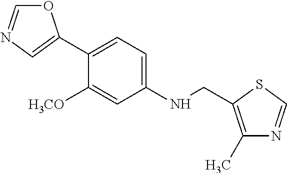 [SMILES: COc1cc(NCc2scnc2C)ccc1-c1cnco1] | C₁₅H₁₅N₃O₂S | 301.36 |
| 55 | N-(thiazol-5-yl methyl)-3- methoxy-4-(oxazol-5-yl) aniline | 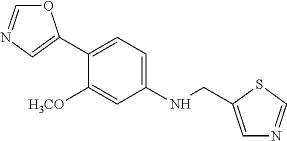 [SMILES: COc1cc(NCc2cncs2)ccc1-c1cnco1] | C₁₄H₁₃N₃O₂S | 287.34 |
| 56 | N-(pyridin-4-yl methyl)-3- methoxy-4-(oxazol-5-yl) aniline | 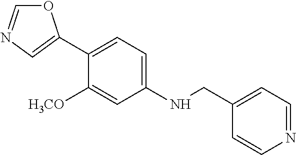 [SMILES: COc1cc(NCc2ccncc2)ccc1-c1cnco1] | C₁₆H₁₅N₃O₂ | 281.31 |
| 57 | N-(6-methylpyridin-2-yl methyl)- 3-methoxy-4-(oxazol-5-yl) aniline | 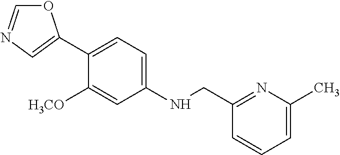 [SMILES: COc1cc(NCc2cccc(C)n2)ccc1-c1cnco1] | C₁₇H₁₇N₃O₂ | 295.34 |
| 58 | 2-methoxy-4-(oxazol-5-yl)-5- [(quinolin-4-ylmethyl)-amino] phenol | 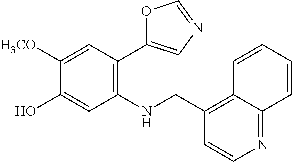 [SMILES: COc1cc(-c2cnco2)c(NCc2ccnc3ccccc23)cc1O] | C₂₀H₁₇N₃O₃ | 347.37 |
| 59 | N-(2-chloropyridin-3-ylmethyl)- 3-methoxy-4-(oxazol-5-yl) aniline | 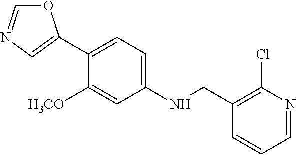 [SMILES: COc1cc(NCc2cccnc2Cl)ccc1-c1cnco1] | C₁₆H₁₄ClN₃O₂ | 315.76 |
| 60 | N-(6-methoxy-pyridin-3-ylmethyl)- 3-methoxy-4-(oxazol-5-yl) aniline | 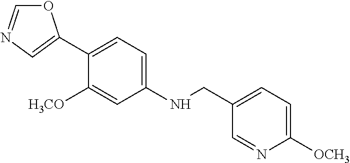 [SMILES: COc1ccc(CNc2ccc(-c3cnco3)c(OC)c2)cn1] | C₁₇H₁₇N₃( )₃ | 311.34 |
| 61 | N-(4-methoxybenzyl)-3- methoxy-4-(oxazol-5-yl) aniline | 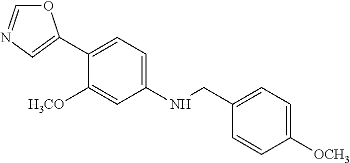 [SMILES: COc1ccc(CNc2ccc(-c3cnco3)c(OC)c2)cc1] | C₁₈H₁₈N₂O₃ | 310.35 |
| 62 | N-(3-phenylpropen-2-yl)-3- methoxy-4-(oxazol-5-yl) aniline | 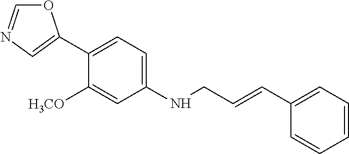 [SMILES: COc1cc(NC/C=C/c2ccccc2)ccc1-c1cnco1] | C₁₉H₁₈N₂O₂ | 3O6.36 |
| 63 | N-[3-(2-methoxyphenylpropen- 2-yl]-3-methoxy-4-(oxazol-5- yl) aniline | 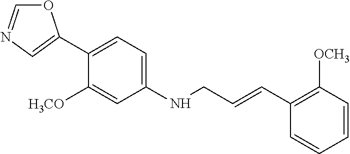 [SMILES: COc1ccccc1/C=C/CNc1ccc(-c2cnco2)c(OC)c1] | C₂₀H₂₀N₂O₃ | 336.39 |
| 64 | N-(4-Fluorophenylmethylidene)- 3-methoxy-4-(oxazol-5-yl)- aniline | 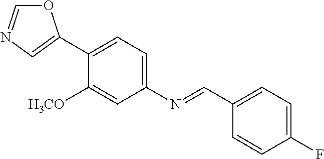 [SMILES: COc1cc(/N=C/c2ccc(F)cc2)ccc1-c1cnco1] | C₁₇H₁₃FN₂O₂ | 296.30 |
| 65 | N-(phenylpropen-1-ylidene)-3- methoxy-4-(oxazol-5-yl)-aniline | 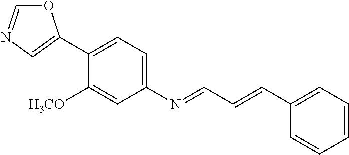 [SMILES: COc1cc(/N=C/C=C/c2ccccc2)ccc1-c1cnco1] | C₁₉H₁₆N₂O₂ | 3O4.35 |
| 66 | (4-Methoxy-2-methylphenyl) hydrazonomalononitrile | 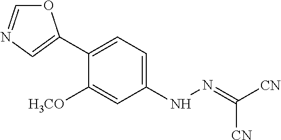 [SMILES: COc1cc(NN=C(C#N)C#N)ccc1-c1cnco1] | C₁₃H₉N₅O₂ | 267.25 |
| 67 | 2-methoxy-5-(oxazol-5-yl) aniline | 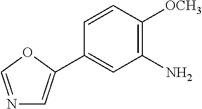 [SMILES: COc1ccc(-c2cnco2)cc1N] | C₁₀H₁₀N₂O₂ | 190.20 |
| 68 | N-[3-methoxy-4-(oxazol-5-yl)- phenyl methanesulfonamide | 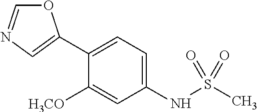 [SMILES: COc1cc(NS(C)(=O)=O)ccc1-c1cnco1] | C₁₁H₁₂N₂O₄S | 268.29 |
| 69 | N-[3-methoxy-4-(oxazol-5-yl)- phenyl toluenesulfonamide | 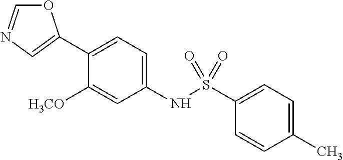 [SMILES: COc1cc(NS(=O)(=O)c2ccc(C)cc2)ccc1-c1cnco1] | C₁₇H₁₆N₂O₄S | 344.39 |
| 70 | 3-[3-methoxy-4-(oxazol-5-yl)- anilino]-1-propanesulfonic acid | 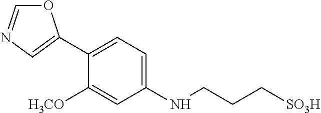 [SMILES: COc1cc(NCCCS(=O)(=O)O)ccc1-c1cnco1] | C₁₃H₁₆N₂O₅S | 312.34 |
| 71 | N-(3,7-dimethyl-octa-2,6-dienyl)- 3-methoxy-4-(oxazol-5-yl) aniline | 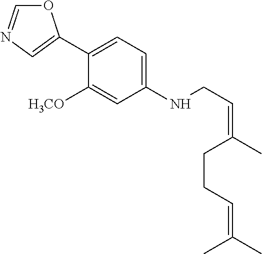 [SMILES: COc1cc(NC/C=C(/C)CCC=C(C)C)ccc1-c1cnco1] | C₂₀H₂₆N₂O₂ | 326.44 |
| 72 | N-[2-(5-methylthien-2-yl)-ethyl]- 3-methoxy-4-(oxazol-5-yl) aniline | 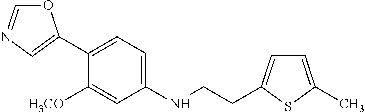 [SMILES: COc1cc(NCCc2ccc(C)s2)ccc1-c1cnco1] | C₁₇H₁₈N₂O₂S | 314.40 |
| 73 | N-[2-(5-bromothien-2-yl)-ethyl]- 3-methoxy-4-(oxazol-5-yl) aniline | 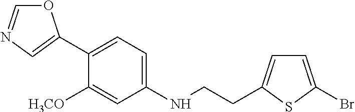 [SMILES: COc1cc(NCCc2ccc(Br)s2)ccc1-c1cnco1] | C₁₆H₁₅BrN₂O₂S | 379.27 |
| 74 | N-(1,2-benzisoxazol-3-ylethyl)- 3-methoxy-4-(oxazol-5-yl) aniline | 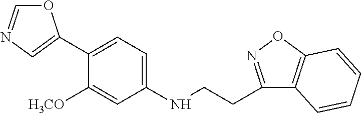 [SMILES: COc1cc(NCCc2noc3ccccc23)ccc1-c1cnco1] | C₁₉H₁₇N₃O₃ | 335.36 |
| 75 | N-(dibenzofuran-4-ylmethyl)- 3-methoxy-4-(oxazol-5-yl) aniline | 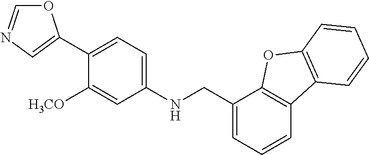 [SMILES: COc1cc(NCc2cccc3c2oc2ccccc23)ccc1-c1cnco1] | C₂₃H₁₈N₂O₃ | 370.41 |
| 76 | N-(5-methylthien-2-ylmethyl)- 3-methoxy-4-(oxazol-5-yl) benzylamine | 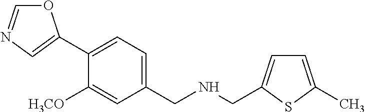 [SMILES: COc1cc(CNCc2ccc(C)s2)ccc1-c1cnco1] | C₁₇H₁₈N₂O₂S | 314.40 |
| 77 | N-(5-chlorothien-2-ylmethyl)-3- methoxy-4-(oxazol-5-yl) benzylamine | 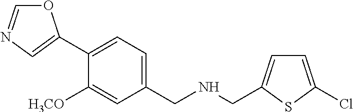 [SMILES: COc1cc(CNCc2ccc(Cl)s2)ccc1-c1cnco1] | C₁₆H₁₅ClN₂O₂S | 334.82 |
| 78 | N-(benzo[1,2,5] oxadiazol-4- ylmethyl)-3-methoxy-4(oxazol-5-yl) benzylamine | 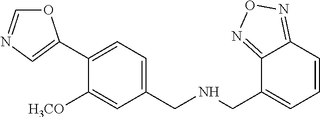 [SMILES: COc1cc(CNCc2cccc3nonc23)ccc1-c1cnco1] | C₁₈H₁₆N₄O₃ | 336.35 |
| 79 | 1-(2-methyl-5-oxazol-5-yl anilino) methyl-naphthol-2 | 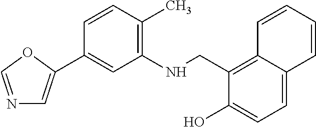 [SMILES: Cc1ccc(-c2cnco2)cc1NCc1c(O)ccc2ccccc12] | C₂₁H₁₈N₂O₂ | 330.39 |
| 80 | N-(2-benzothien-2-yl-ethyl)-N- (3-methoxy-4-oxazol-5-yl)-2- bromopropionamide | 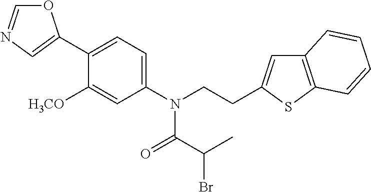 [SMILES: COc1cc(N(CCc2cc3ccccc3s2)C(=O)C(C)Br)ccc1-c1cnco1] | C₂₃H₂₁BrN₂O₃S | 485.40 |
| 81 | N-[2-(4-chlorocinnolin-3-yl)- ethyl]-3-methoxy-4-(oxazol-5-yl) aniline | 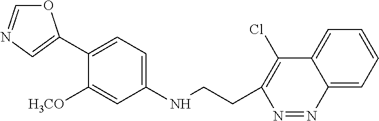 [SMILES: COc1cc(NCCc2nnc3ccccc3c2Cl)ccc1-c1cnco1] | C₂₀H₁₇ClN₄O₂ | 380.83 |
| 82 | N-[2-(5-chlorobenzathien-2-yl)- ethyl]-3-methoxy-4-(oxazol-5-yl) aniline | 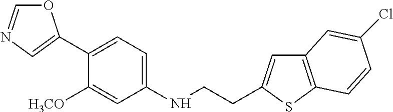 [SMILES: COc1cc(NCCc2cc3cc(Cl)ccc3s2)ccc1-c1cnco1] | C₂₀H₁₇ClN₂O₂S | 384.88 |
| 83 | N-(4,6-dichloro-2H-sulfurbenzopyran- 3-ylmethyl)-3-methoxy-4- (oxazol-5-yl) aniline | 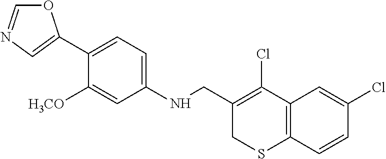 [SMILES: COc1cc(NCC2=C(Cl)c3cc(Cl)ccc3SC2)ccc1-c1cnco1] | C₂₀H₁₆Cl₂N₂O₂S | 419.33 |
| 84 | N-(3-methoxy-4-oxazol-5-yl-benzyl)- N-(5-methyl-benzothien-2- ylmethyl)-methylsulfonamide | 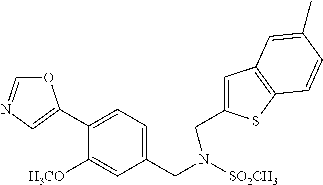 [SMILES: COc1cc(CN(Cc2cc3cc(C)ccc3s2)S(C)(=O)=O)ccc1-c1cnco1] | C₂₂H₂₂N₂O₄S₂ | 442.55 |
| 85 | N-(benzothien-3-ylmethyl)-3- methoxy-4-(oxazol-5-yl) benzylamine | 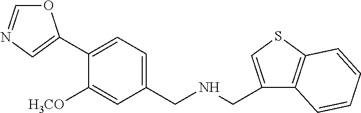 [SMILES: COc1cc(CNCc2csc3ccccc23)ccc1-c1cnco1] | C₂₀H₁₈N₂O₂S | 350.44 |
| 86 | N-(1-methyl-1H-indazole-5-methyl)- 3-methoxy-4-(oxazol-5-yl) benzylamine | 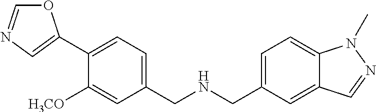 [SMILES: COc1cc(CNCc2ccc3c(cnn3C)c2)ccc1-c1cnco1] | C₂₀H₂₀N₄O₂ | 384.40 |
| 87 | N-methyl-N-(5-chlorobenzothien- 3-ylmethyl)-3-methoxy-4- (oxazol-5-yl) benzylamine | 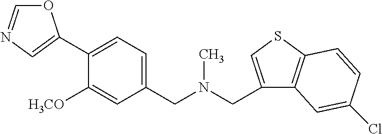 [SMILES: COc1cc(CN(C)Cc2csc3ccc(Cl)cc23)ccc1-c1cnco1] | C₂₁H₁₉ClN₂O₂S | 398.91 |
| 88 | N-(10-methyl-10H-phenothiazin-3- ylmethyl)-3-methoxy-4-oxazol- 5-yl aniline | 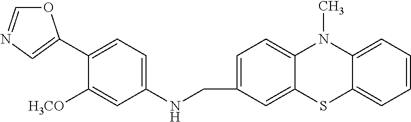 [SMILES: COc1cc(NCc2ccc3c(c2)Sc2ccccc2N3C)ccc1-c1cnco1] | C₂₄H₂₁N₃O₂S | 415.51 |
| 89 | N-(5-methylthien-2-ylmethyl)-3- chloride-4-oxazol-5-yl benzylamine | 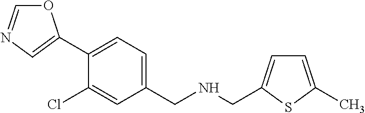 [SMILES: Cc1ccc(CNCc2ccc(-c3cnco3)c(Cl)c2)s1] | C₁₆H₁₅ClN₂OS | 318.82 |
| 90 | N-dibenzothien-4-ylmethyl-3- chloro-4-oxazol-5-yl aniline | 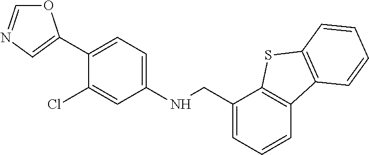 [SMILES: Clc1cc(NCc2cccc3c2sc2ccccc23)ccc1-c1cnco1] | C₂₂H₁₅ClN₂OS | 390.89 |
| 91 | N-(benzo[1,2,5]-thiadiazol-4- ylmethyl)-3-chloro-4-oxazol-5-yl benzylamine | 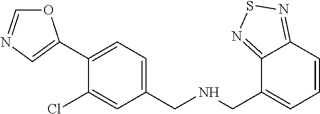 [SMILES: Clc1cc(CNCc2cccc3nsnc23)ccc1-c1cnco1] | C₁₇H₁₃ClN₄OS | 356.83 |
| 92 | 3-(3-methoxy-4-oxazol-5-yl anilino) methyl-4-H-benzopyran-6-ol | 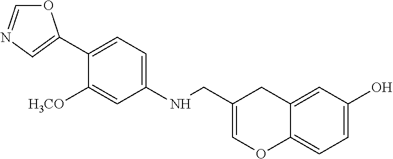 [SMILES: COc1cc(NCC2=COc3ccc(O)cc3C2)ccc1-c1cnco1] | C₂₀H₁₈N₂O₄ | 350.37 |
| 93 | N-(5-bromofuran-2-ylmethyl)-3- methoxy-4-(oxazol-5-yl) benzylamine | 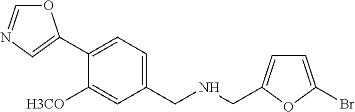 [SMILES: COc1cc(CNCc2ccc(Br)o2)ccc1-c1cnco1] | C₁₆H₁₅BrN₂O₃ | 363.21 |
| 94 | N-(2-methylacridine-9-methyl)- 3-methoxy-4-(oxazol-5-yl) aniline | 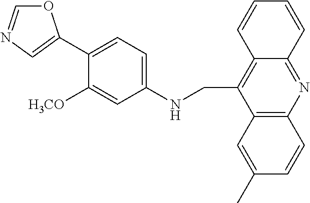 [SMILES: COc1cc(NCc2c3ccccc3nc3ccc(C)cc23)ccc1-c1cnco1] | C₂₅H₂₁N₃O₂ | 395.46 |
| 95 | N-(anthracen-9-yl methyl) 3- methoxy-4-(oxazol-5-yl) aniline | 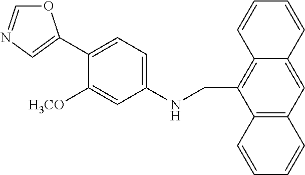 [SMILES: COc1cc(NCc2c3ccccc3cc3ccccc23)ccc1-c1cnco1] | C₂₅H₂₀N₂O₂ | 380.45 |
| 96 | 5-(3-methoxy-4-oxazole-5-yl- anilinomethyl furan-2-yl methanol | 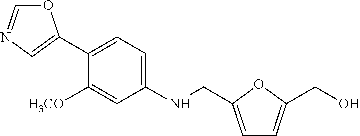 [SMILES: COc1cc(NCc2ccc(CO)o2)ccc1-c1cnco1] | C₁₆H₁₆N₂O₄ | 300.31 |
| 97 | N-(4-bromo-5-ethylthien-2-ylmethyl) 3-methoxy-4-(oxazol-5-yl) aniline | 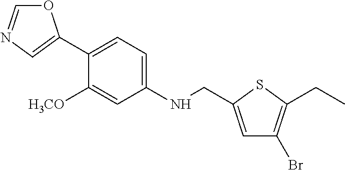 [SMILES: CCc1sc(CNc2ccc(-c3cnco3)c(OC)c2)cc1Br] | C₁₇H₁₇BrN₂O₂S | 393.30 |
| 98 | N-(thien-2-yl sulfonyl)-3-methoxy- 4-oxazole-5-yl aniline | 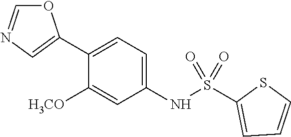 [SMILES: COc1cc(NS(=O)(=O)c2cccs2)ccc1-c1cnco1] | C₁₄H₁₂N₂O₄S₂ | 336.38 |
| 99 | N-(5-chloro-4-nitro-thien-2-yl sulfonyl)-3-methoxy-4-oxazole-5-yl aniline | 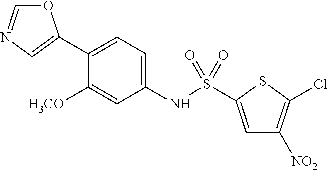 [SMILES: COc1cc(NS(=O)(=O)c2cc([N+](=O)[O-])c(Cl)s2)ccc1-c1cnco1] | C₁₄H₁₀ClN₃O₆S₂ | 415.82 |
| 100 | N-(2,3-dihydobenzofuro-5-yl methyl)- 3-methoxy-4-(oxazol-5-yl) aniline | 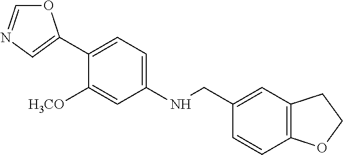 [SMILES: COc1cc(NCc2ccc3c(c2)CCO3)ccc1-c1cnco1] | C₁₉H₁₈N₂O₃ | 322.36 |
| 101 | N-(5-acetyl thien-2-yl methyl)-3- methoxy-4-oxazole-5-yl aniline | 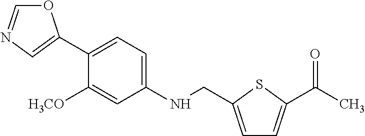 [SMILES: COc1cc(NCc2ccc(C(C)=O)s2)ccc1-c1cnco1] | C₁₇H₁₆N₂O₃S | 328.39 |
| 102 | N-(5-methylthiothien-2-ylmethyl)- 3-methoxy-4-(oxazol-5-yl) aniline | 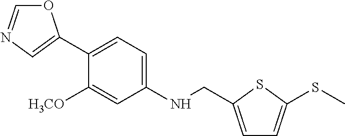 [SMILES: COc1cc(NCc2ccc(SC)s2)ccc1-c1cnco1] | C₁₆H₁₆N₂O₂S₂ | 332.44 |
| 103 | N-[5-(4-hydroxylpiperidinyl)-thien- 2-yl]-3-methoxy-4-oxazole-5- yl aniline | 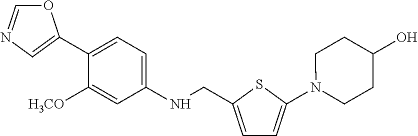 [SMILES: COc1cc(NCc2ccc(N3CCC(O)CC3)s2)ccc1-c1cnco1] | C₂₀H₂₃N₃O₃S | 385.48 |
| 104 | N-[5-(2-chloro-4- trifluoromethylphenyl)furan- 2-ylmethyl]-3- methoxy-4-oxazol-5-yl aniline | 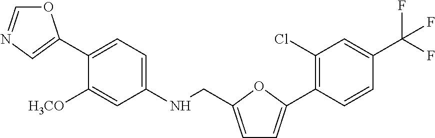 [SMILES: COc1cc(NCc2ccc(-c3ccc(C(F)(F)F)cc3Cl)o2)ccc1-c1cnco1] | C₂₂H₁₆ClF₃N₂O₃ | 448.83 |
| 105 | N-(furan-3-yl propen-1-yl)-3- methoxy-4-oxazol-5-yl aniline | 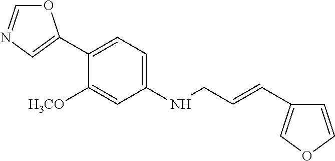 [SMILES: COc1cc(NC/C=C/c2ccoc2)ccc1-c1cnco1] | C₁₇H₁₆N₂O₃ | 296.33 |
| 106 | N-(benzothien-5-yl methyl)-3- methoxy-4-oxazol-5-yl aniline | 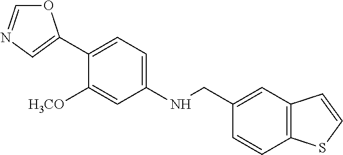 [SMILES: COc1cc(NCc2ccc3sccc3c2)ccc1-c1cnco1] | C₁₉H₁₆N₂O₂S | 336.41 |

Another aspect the present invention provides an anti-viral, anti-tumor composition comprising a therapeutically effective amount of the above-mentioned phenyl-oxazolyl derivative or a pharmaceutically acceptable salt thereof, as well as pharmaceutically acceptable excipients. The compounds themselves, or in combination with officinal excipients, diluents and the like can be orally administered in the form of tablets, capsules, granules, powders or syrups, or in the form of an injection for parenteral administration.

The above formulations can be prepared by conventional methods of pharmaceutical procedures. Examples of pharmaceutically acceptable adjuvants that can be used include excipients (e.g. sugar derivatives such as lactose, sucrose, glucose, mannitol and sorbitol; starch derivatives such as corn starch, potato starch, dextrin and carboxymethyl starch; cellulose derivatives such as crystalline cellulose, hydroxypropyl cellulose, hydroxymethyl cellulose, hydroxymethyl cellulose calcium, carboxymethyl cellulose sodium; acacia; dextran; silicate derivatives such as magnesium aluminum metasilicate, phosphate derivatives such as calcium phosphate; carbonate derivatives such as calcium carbonate; sulfate derivatives such as calcium sulfate; binders (e.g., gelatin, polyvinyl pyrrolidone, and polyethylene glycol); disintegrants (for example, cellulose derivatives such as carboxymethyl cellulose sodium, polyvinyl pyrrolidone); lubricants (such as talc, calcium stearate, magnesium stearate, spermaceti, boric acid, sodium benzoate, leucine), stabilizers (methylparaben, propylparaben, etc.); correctan (e.g., commonly used sweeteners, sour agents and perfumes); liquid diluents and solvents for injection (e.g., water, ethanol and glycerol and the like).

The present invention also provides the results of experimental studies on the biological activity of said compounds. IMPDH inhibitory activity of the compounds of the present invention were measured (Table 2); for some of the compounds, mouse spleen cell proliferation inhibiting activity were also carried out, wherein the IC₅₀ value of compound 63 is 2.22 μg/mL.

TABLE 2
IMPDH inhibition activity of the compounds

| Number | inhibition rate (%) 10 μg/mL |
|---|---|
| 1 | 65.7 |
| 2 | 68.7 |
| 4 | 74.9 |
| 5 | 58.9 |
| 9 | 38.1 |
| 11 | 69.6 |
| 20 | 47.7 |
| 42 | 60.7 |
| 53 | 39.2 |
| 56 | 37.4 |
| 57 | 45.1 |
| 61 | 73.7 |
| 65 | 32.0 |

Progress in the studies on the antiviral drugs targeting IMPDH shows that IMPDH inhibitors demonstrate broad-spectrum antiviral activity, they showed strong inhibitory effects against hepatitis B virus, hepatitis C virus, influenza virus, human cytomegalovirus, respiratory syncytial virus, type 1 herpes simplex virus, encephalomyocarditis virus, Venezuelan equine encephalitis virus. In cell stain 2215 as an in vitro experimental model cell line, TC₅₀ (half toxic concentration), IC₅₀ (50% inhibition concentration of the virus) and SI (selective index, SI=TC₅₀/IC₅₀) values obtained from Compound 4 against hepatitis B virus were 100 μg/mL, 2.72 μg/mL and 36.76 μg/mL, respectively; and those of the compound against hepatitis B virus strain A64 (lamivudine resistance) were>50 μg/mL, 1.78 μg/mL and >28.08, respectively.

With Vero (African green monkey kidney) cells as viral hosts, inhibitory effects of the compounds of present invention on the grades of pathological lesions by coxsackievirus B3 (COX-B3), B6 (COX-B6) viruses in Vero cells were determined. The measured results of viral inhibitory activity of the compounds are shown in Table 3 and Table 4; wherein RBV is the control drug ribavirin. The inhibitory activities of the measured compounds against coxsackievirus B3, B6 were stronger than the control.

TABLE 3
Anti-Coxsackie virus type B3 (COX-
B3) activities of the compounds

| No. | IC₅₀ |
|---|---|
| 1 | 0.44 |
| 2 | 0.23 |
| 3 | 0.35 |
| 4 | 0.44 |
| 5 | 0.47 |
| 6 | 0.47 |
| 7 | >66.67 |
| 8 | 0.45 |
| 9 | 12.83 |
| 10 | 0.27 |
| 11 | 0.44 |
| 12 | 0.23 |
| 13 | 0.30 |
| 14 | 0.44 |
| 15 | 0.46 |
| 16 | 0.45 |
| 17 | 2.56 |
| 18 | 0.25 |
| 19 | 12.83 |
| 20 | 0.64 |
| 21 | 2.47 |
| 22 | 0.82 |
| 23 | 0.76 |
| 24 | 0.57 |
| 25 | 0.53 |
| 26 | 0.57 |
| 27 | 1.47 |
| 28 | 1.23 |
| 29 | 10.68 |
| 30 | 0.54 |
| 31 | 0.88 |
| 32 | 0.68 |
| 33 | 0.64 |
| 34 | 0.57 |
| 35 | 0.57 |
| 36 | 0.59 |
| 37 | 0.63 |
| 38 | 0.88 |
| 39 | 11.12 |
| 40 | 0.26 |
| 41 | 0.63 |
| 42 | 1.43 |
| 43 | 1.03 |
| 44 | 0.69 |
| 45 | 0.88 |
| 46 | 0.76 |
| 47 | 0.56 |
| 48 | 0.50 |
| 49 | 0.82 |
| 50 | 0.46 |
| 51 | 2.47 |
| 52 | 3.25 |
| 53 | >2.47 |
| 54 | 1.43 |
| 55 | 1.88 |
| 56 | 5.75 |
| 57 | 2.47 |
| 58 | 3.22 |
| 59 | 1.90 |
| 60 | 0.57 |
| 61 | 0.64 |
| 62 | 0.63 |
| 63 | 0.47 |
| 64 | 4.28 |
| 65 | 1.23 |
| 66 | 1.43 |
| 67 | 1.88 |
| 68 | 1.53 |
| 69 | 0.98 |
| 70 | 155.21 |
| 71 | 1.25 |
| 72 | 0.44 |
| 73 | 0.35 |
| 74 | 0.63 |
| 75 | 0.45 |
| 76 | 0.24 |
| 77 | 0.35 |
| 78 | 0.36 |
| 79 | 0.35 |
| 80 | 0.24 |
| 81 | 0.29 |
| 82 | 0.33 |
| 83 | 0.42 |
| 84 | 0.34 |
| 85 | 0.44 |
| 86 | 0.34 |
| 87 | 0.35 |
| 88 | 0.36 |
| 89 | 0.26 |
| 90 | 0.26 |
| 91 | 0.28 |
| 92 | 0.63 |
| 93 | 0.88 |
| 94 | 0.68 |
| 95 | 0.55 |
| 96 | 1.43 |
| 97 | 0.23 |
| 98 | 0.88 |
| 99 | 0.78 |
| 100 | 0.46 |
| 101 | 0.48 |
| 102 | 0.35 |
| 103 | 0.40 |
| 104 | 0.45 |
| 105 | 1.23 |
| 106 | 0.44 |
| RBV | 222.22 |

Note:
IC₅₀: μg/mL.

TABLE 4
Anti-Coxsackie virus type B6 (COX-
B6) activities of the compounds

| No. | IC₅₀ |
|---|---|
| 1 | 0.44 |
| 2 | 0.63 |
| 3 | 1.43 |
| 4 | 0.27 |
| 5 | 0.44 |
| 6 | 0.27 |
| 7 | 18.50 |
| 9 | 12.83 |
| 10 | 0.36 |
| 11 | 1.33 |
| 20 | 0.82 |
| 21 | 3.25 |
| 22 | 3.83 |
| 41 | 1.88 |
| 42 | 1.93 |
| 49 | 1.53 |
| 52 | >2.47 |
| 53 | 7.41 |
| 54 | 3.18 |
| 55 | 3.25 |
| 56 | 4.28 |
| 57 | 1.88 |
| 59 | 1.05 |
| 60 | 3.15 |
| 61 | 1.06 |
| 62 | 1.43 |
| 63 | 2.47 |
| 64 | 17.24 |
| 65 | 5.75 |
| 66 | 4.28 |
| 68 | 7.41 |
| 71 | >0.82 |
| RBV | 222.22 |

Note:
IC₅₀: μg/mL.

The compounds of the invention also exhibit strong inhibitory activities against HCV, influenza viruses.

IMPDH is a key enzyme in purine biosynthesis, it catalyzes oxidation of inosinic acid (IMP) to xanthylic acid (XMP), and then under the catalysis of CMP synthase, XMP transforms into guanylic acid (GMP). CMP is an important enzyme substrate for DNA and RNA synthesis and plays an important role in the cell growth and differentiation, apoptosis as well as in cell signaling. IMPDH inhibition will lead to a lack of GMP, and further, to the blockade of DNA synthesis, leading to the cells in a standstill in G1 phase, thus catalytic reaction of IMPDH are closely related to cell proliferation. Therefore, IMPDH inhibitors have broad-spectrum anti-tumor activities.

According to the progress in the studies on anticancer drugs using IMPDH as a target, the present invention measured inhibitory activities of the compounds of present invention against human hepatoma cell HepG2 and human lung adenocarcinoma cell A549, respectively, the measured results are shown in Table 5:

TABLE 5
The effect of compounds on tumor cells in vitro

| | HepG2 | A549 |
|---|---|---|
| No. | IC₅₀ | IC₅₀ |
| 1 | 0.95 | NT |
| 2 | 7.952 | 18.59 |
| 3 | 20.92 | 10.74 |
| 4 | 29.09 | 16.03 |
| 5 | 83.43 | 16.51 |
| 6 | 31.75 | 16.89 |
| 7 | >100 | >100 |
| 10 | 21.69 | 26.75 |
| 11 | 42.08 | 29.67 |
| 20 | 2.883 | 11.60 |
| 21 | >100 | 122.2 |
| 22 | 9.905 | 26.19 |
| 41 | 39.97 | 22.75 |
| 42 | 31.32 | 20.70 |
| 49 | 0.8969 | 4.838 |
| 53 | 22.54 | 38.56 |
| 54 | 53.32 | 29.48 |
| 55 | 42.13 | 31.92 |
| 57 | 42.78 | 36.67 |
| 61 | 36.20 | 29.41 |
| 62 | >100 | 32.04 |
| 63 | 19.35 | 14.14 |
| Cisplatin | 104.1 | 263.4 |

Note:
IC₅₀: μg/mL, NT: not detected.

The results showed above preliminarily verified the inhibitory activities of compounds or pharmaceutically acceptable salts thereof in this invention against various cancer cells, thereby laying foundation for their development and application as anticancer drugs.

With reference to specific examples below, the present invention is further depicted, advantages and features of the invention will become apparent with the descriptions. However, these embodiments are merely exemplary, and they do not constitute any limitation to the scope of the present invention. The skilled practitioners of this field will appreciate that, without departing from the spirit and scope of the present invention, the details and form of the present technical schemes of this invention can be modified or replaced, but such modifications and alterations fall within the scope of the protection of the present invention.

### EMBODIMENTS

### Example 1

### Synthesis of N-(thien-2-ylmethyl)-3-methoxy-4-(oxazol-5-yl) aniline (1)

Dissolve 3-methoxy-4-(oxazol-5-yl) aniline (190.2 mg, 1 mmol) in absolute ethanol (3 ml) in a 25 ml flask, add in 2-thiophene carbaldehyde (1.1 mmol), stir at room temperature until the starting material 3-methoxy-4-(oxazol-5-yl) aniline disappears. Add a reducing agent (e.g. NaBH₄, 2 mmol) at 0-10° C., warm at ambient temperature until the disappearance of the intermediate. Extract with dichloromethane (20 ml×3) add in 10% HCl to remove excessive NaBH₄, then basify the solution with aqueous ammonia, wash the solution with water until neutral. Dessicate the solution over anhydrous Na₂SO₄. Filter and evaporate the solvent from the solution. Flash isolation to obtain 165 mg product 1 (57.6%) as a yellow solid.

¹H NMR (CDC₃, δ) 3.89 (s, 3H, —OCH₃), 4.57 (s, 2H, —CH₂—), 6.28 (s, 1H, 2-Ph), 6.37 (d, J=8.5 Hz, 1H, 6-Ph), 6.98 (m, 1H, 3-Th), 7.04 (m, 1H, 4-Th), 7.24 (m, 1H, 5-Th), 7.34 (s, 1H, 4-Ox), 7.58 (d, J=8.5 Hz, 1H, 5-Ph), 7.82 (s, 1H, 2-Ox).

### Example 2

### Synthesis of N-(5-methylthien-2-ylmethyl)-3-methoxy-4-(oxazol-5-yl) aniline (2)

Use 3-methoxy-4-(oxazol-5-yl) aniline and 5-methyl-thiophene-2-aldehyde as starting materials, follow the similar procedures applied in Example 1 to obtain compound 2, yield: 74.0%.

¹H NMR (CDC₃, δ) 2.45 (s, 3H, CH₃—Th), 3.89 (s, 3H, —OCH₃), 4.29 (br, 1H, NH), 4.47 (s, 2H, —CH₂—), 6.26 (s, 1H, 2-Ph), 6.35 (d, j=8.5 Hz, 1H, 6-Ph), 6.60 (d, J=2.5 Hz, 1H, 4-Th), 6.80 (d, J=3.0 Hz, 1H, 3-Th), 7.33 (s, 1H, 4-Ox), 7.57 (d, J=8.5 Hz, 1H, 5-Ph), 7.81 (s, 1H, 2-Ox).

### Example 3

### Synthesis of N-(5-ethylthien-2-ylmethyl)-3-methoxy-4-(oxazol-5-yl) aniline (3)

Use 3-methoxy-4-(oxazol-5-yl) aniline and 5-ethyl-thiophene-2-carbaldehyde as starting materials, follow the similar procedures applied in Example 1 to obtain compound 3, yield: 85.9%.

¹H NMR (CDC₃, δ) 1.29 (t, 3H, —CH₂CH₃), 2.81 (q, 2H, —CH₂CH₃), 3.89 (s, 3H, —OCH₃), 4.48 (s, 2H, —CH₂—), 6.26 (s, 1H, 2-Ph), 6.35 (d, J=8.5 Hz, 1H, 6-Ph), 6.64 (d, J=3.5 Hz, 1H, 4-Th), 6.82 (d, J=3.5 Hz, 1H, 3-Th), 7.34 (s, 1H, 4-Ox), 7.57 (d, J=8.5 Hz, 1H, 5-Ph), 7.81 (s, 1H, 2-Ox).

### Example 4

### Synthesis of N-(5-chlorothien-2-ylmethyl)-3-methoxy-4-(oxazol-5-yl) aniline (4)

Use 3-methoxy-4-(oxazol-5-yl) aniline and 5-chloro-thiophene-2-carbaldehyde as starting materials, follow the similar procedures applied in Example 1 to obtain compound 4, yield: 76.9%.

¹H NMR (CDC₃, δ) 3.89 (s, 3H, —OCH₃), 4.47 (s, 2H, —CH₂—), 6.25 (s, 1H, 2-Ph), 6.34 (d, J=8.5 Hz, 1H, 6-Ph), 6.77 (d, J=4 Hz, 1H, 3-Th), 6.80 (d, J=4 Hz 1H, 4-Th), 7.34 (s, 1H, 4-Ox), 7.58 (d, J=8.5 Hz, 1H, 5-Ph), 7.82 (s, 1H, 2-Ox).

### Example 5

### Synthesis of N-(5-bromothien-2-ylmethyl)-3-methoxy-4-(oxazol-5-yl) aniline (5)

Use 3-methoxy-4-(oxazol-5-yl) aniline and 5-bromo-thiophene-2-carbaldehyde as starting materials, follow the similar procedures applied in Example 1 to obtain compound 5, yield: 56.2%.

¹H NMR (CDC₃, δ) 3.91 (s, 3H, —OCH₃), 4.49 (s, 2H, —CH₂—), 6.25 (s, 1H, 2-Ph), 6.35 (d, J=8.5 Hz, 1H, 6-Ph), 6.79 (d, J=3.5 Hz, 1H, 3-Th), 6.91 (d, J=4 Hz, 1H, 4-Th), 7.35 (s, 1H, 4-Ox), 7.58 (d, J=8.5 Hz, 1H, 5-Ph), 7.82 (s, 1H, 2-Ox).

### Example 6

### N-(5-phenylthien-2-ylmethyl)-3-methoxy-4-(oxazol-5-yl) aniline (6)

Use 3-methoxy-4-(oxazol-5-yl) aniline and 5-phenyl-thiophene-2-carbaldehyde as starting materials, follow the similar procedures applied in Example 1 to obtain compound 6, yield: 72.1%.

¹H NMR (CDC₃, δ) 3.90 (s, 3H, —OCH₃), 4.56 (s, 2H, —CH₂—), 6.29 (s, 1H, 2-Ph), 6.38 (d, J=8.5 Hz, 1H, 6-Ph), 6.99 (d, J=3.5 Hz, 1H, 3-Th), 7.17 (d, J=3.5 Hz, 1H, 4-Th), 7.27 (t, J=7.5 Hz, J=7.5 Hz, 1H, 4′-Ph), 7.34 (s, 1H, 4-Ox), 7.36 (t, J=7.5 Hz, J=7.5 Hz, 2H, 3′, 5′-Ph), 7.56 (d, J=7.5 Hz, 2H, 2′, 6′-Ph), 7.59 (d, J=8.5 Hz, 1H, 5-Ph), 7.82 (s, 1H, 2-Ox).

### Example 7

### Synthesis of 5-(3-methoxy-4-oxazol-5-yl-anilinomethyl)-thien-2-yl carboxylic acid (7)

Dissolve 3-methoxy-4-(oxazol-5-yl) aniline (190.2 mg, 1 mmol) in absolute ethanol (3 ml), add in 5-formyl-2-thiophene-carboxylic acid (1.1 mmol), stir at room temperature until the starting material 3-methoxy-4-(oxazol-5-yl) aniline disappears. Add a reducing agent (e.g. NaBH₄, 2 mmol) at 0-10° C., then warm at ambient temperature until the disappearance of the intermediate. Dissolve the mixture under heating with ethanol, filter, and evaporate part of the solvent, let stand the mixture to obtain a yellow solid product 7, yield: 54.6%.

¹H NMR (DMSO-d6, δ) 3.82 (s, 3H, —OCH₃), 4.39 (br, 1H, —NH), 4.40 (s, 2H, —CH₂—), 6.32 (d, J=8.5 Hz, 1H, 6-Ph), 6.39 (s, 1H, 2-Ph), 6.85 (d, J=3 Hz, 1H, 3-Th); 7.03 (d, J=3.5 Hz, 1H, 4-Th), 7.18 (s, 1H, 4-Ox), 7.37 (d, J=8.5 Hz, 1H, 5-Ph); 8.21 (s, 1H, 2-Ox).

### Example 8

### Synthesis of N-(benzothiazol-2-ylmethyl)-3-methoxy-4-(oxazol-5-yl) aniline (8)

Use 3-methoxy-4-(oxazol-5-yl) aniline and benzothiazole-2-carbaldehyde as starting materials, follow the similar procedures applied in Example 1 to obtain compound 8.

¹H NMR (CDC₃, δ) 3.87 (s, 3H, —OCH₃), 4.65 (s, 2H, —CH₂—), 6.32 (s, 1H, 2-Ph), 6.38 (d, J=8.5 Hz, 1H, 6-Ph), 7.54 (m, 2H, 5,6-Thia), 7.33 (s, 1H, 4-Ox), 7.57 (d, J=8.5 Hz, 1H, 5-Ph), 7.81 (s, 1H, 2-Ox), 8.11 (m, 1H, 7-Thia), 8.22 (m, 1H, 4-Thia).

### Example 9

### Synthesis of N-(thien-2-yl methylene)-3-methoxy-4-(oxazol-5-yl) aniline (9)

Dissolve 3-methoxy-4-(oxazol-5-yl) aniline (190.2 mg, 1 mmol) in absolute ethanol (3 ml), add in 2-thiophene carbaldehyde (1.1 mmol), stir at room temperature until starting material 3-methoxy-4-(oxazol-5-yl) aniline disappears. Extract with dichloromethane (20 ml×3), wash with water until the wash solution turns neutral. Dry over anhydrous Na₂SO₄. Filter and evaporate the solvent from the solution, recrystallize the residue with dichloromethanehexane to obtain product 9, yield: 84.5%.

¹H NMR (CDC₃, δ) 4.00 (s, 3H, —OCH₃), 6.90 (s, 1H, 2-Ph), 6.91 (d, J=10.5 Hz, 1H, 6-Ph), 7.16 (dd, J=4.5 Hz, J=4.5 Hz, 1H, 4-Th), 7.52 (d, J=5 Hz, 1H, 3-Th), 7.55 (d, J=5 Hz, 1H, 5-Th), 7.55 (s, 1H, 4-Ox), 7.78 (d, J=10.5 Hz, 1H, 5-Ph), 7.90 (s, 1H, 2-Ox), 8.63 (s, 1H, N═CH—).

### Example 10

### Synthesis of N-(benzothien-2-ylmethyl)-3-methoxy-4-(oxazol-5-yl) aniline (10)

Use 3-methoxy-4-(oxazol-5-yl) aniline and 2-benzothiophenecarbaldehyde as raw materials, follow the similar procedures applied in Example 1 to obtain compound 10, yield: 35.7%.

¹H NMR (CDC₃, δ) 3.88 (s, 3H, —OCH₃), 4.66 (s, 2H, —CH₂—), 6.31 (s, 1H, 2-Ph), 6.39 (d, J=8.5 Hz, 1H, 6-Ph), 7.25 (s, 1H, 3-Th), 7.28 (m, 1H, 5-Th), 7.33 (m, 1H, 6-Th), 7.34 (s, 1H, 4-Ox), 7.58 (d, J=8.5 Hz, 1H, 5-Ph), 7.70 (n, 1H, 4-Th), 7.78 (m, 1H, 7-Th), 7.82 (s, 1H, 2-Ox).

### Example 11

### Synthesis of N-(thien-3-yl methyl)-3-methoxy-4-(oxazol-5-yl) aniline (11)

Use 3-methoxy-4-(oxazol-5-yl) aniline and 3-thiophene aldehyde as the raw materials, follow the similar procedures applied in Example 1 to obtain compound 11, yield: 62.9%.

¹H NMR (CDC₃, δ) 3.88 (s, 3H, —OCH₃), 4.39 (s, 2H, —CH₂—), 6.24 (s, 1H, 2-Ph), 6.34 (d, J=8.5 Hz, 1H, 6-Ph), 7.09 (d, J=5 Hz, 1H, 4-Th), 7.22 (s, 1H, 2-Th), 7.33 (d, J=5 Hz, 1H, 5-Th), 7.33 (s, 1H, 4-Ox), 7.57 (d, J=8.5 Hz, 1H, 5-Ph), 7.81 (s, 1H, 2-Ox).

### Example 12

### Synthesis of N-methyl-N-(5-methylthien-3-ylmethyl)-3-methoxy-4-(oxazol-5-yl) aniline (12)

Use 3-methoxy-4-(oxazol-5-yl) aniline (1 mmol) and 5-methylthiophene-3-carbaldehyde (1.1 mmol) as starting materials to obtain N-(5-methyl-thien-3-yl methyl) 3-methoxy-4-(oxazol-5-yl) aniline follow the similar procedures applied in Example 1, dissolve the separated product in acetone, dessicate over anhydrous K₂CO₃ (2 mmol), add the solution of methyl iodide (1 mmol) in ethyl acetate while stir slowly, when the raw material disappears, extract with ethyl acetate. Flash separate and purify to obtain compound 12.

¹H NMR (CDC₃, δ) 2.43 (s, 3H, CH₃—Th), 2.86 (s, 3H, CH₃—N), 3.88 (s, 3H, —OCH₃), 4.61 (s, 2H, —CH₂—), 6.28 (s, 1H, 2-Ph), 6.45 (d, J=8.5 Hz, 1H, 6-Ph), 6.87 (s, 1H, 4-Th), 7.20 (s, 1H, 2-Th), 7.34 (s, 1H, 4-Ox), 7.58 (d, J=8.5 Hz, 1H, 5-Ph), 7.81 (s, 1H, 2-Ox).

### Example 13

### Synthesis of N-(5-ethyl-2-methylthien-3-ylmethyl) N-(3-methoxy-4-oxazole-5-phenyl) propyn-2-ylamine (13)

Use 3-methoxy-4-(oxazol-5-yl) aniline and 5-ethyl 2-methylthiophene-3-carbaldehyde as starting materials, following a similar method as used in Example 1 to obtain the compound N-(5-ethyl-2-methyl-3-methyl-thiophene) methoxy-4-(oxazol-5-yl) aniline; subsequently follow the similar procedures applied in Example 12, with a exception of adding 3-bromopropyne instead of iodomethane, to obtain compound 13.

¹H NMR (CDC₃, δ) 1.28 (t, 3H, —CH₂CH₃), 1.81 (s, 2H, —CH₂CCH), 2.41 (s, 3H, CH3), 2.80 (q, 2H, —CH₂CH₃), 3.89 (s, 3H, —OCH₃), 4.09 (s, 2H, —CH₂CCH), 4.60 (s, 2H, —CH₂—), 6.28 (s, 1H, 2-Ph), 6.36 (d, J=8.5 Hz, 1H, 6-Ph), 6.80 (s, 1H, 4-Th), 7.34 (s, 1H, 4-Ox), 7.59 (d, J=8.5 Hz, 1H, 5-Ph), 7.82 (s, 1H, 2-Ox).

### Example 14

### Synthesis of N-(5-chlorothien-3-yl methyl)-3-methoxy-4-(oxazol-5-yl) aniline (14)

Use 3-methoxy-4-(oxazol-5-yl) aniline and 5-chloro-thiophene-3-carbaldehyde as starting materials, follow the similar procedures applied in Example 1 to obtain compound 14.

¹H NMR (CDC₃, δ) 3.90 (s, 3H, —OCH₃), 4.38 (s, 2H, —CH₂—), 6.27 (s, 1H, 2-Ph), 6.35 (d, J=8.5 Hz, 1H, 6-Ph), 6.89 (s, 1H, 4-Th), 7.25 (s, 1H, 2-Th), 7.34 (s, 1H, 4-Ox), 7.58 (d, J=8.5 Hz, 1H, 5-Ph), 7.82 (s, 1H, 2-Ox).

### Example 15

### Synthesis of N-(5-bromothien-3-yl methyl)-3-methoxy-4-(oxazol-5-yl) aniline (15)

Use 3-methoxy-4-(oxazol-5-yl) aniline and 5-bromo-thiophene-3-carbaldehyde as starting materials, follow the similar procedures applied in Example 1 to obtain compound 15.

¹H NMR (CDC₃, δ) 3.91 (s, 3H, —OCH₃), 4.39 (s, 2H, —CH₂—), 6.26 (s, 1H, 2-Ph), 6.34 (d, J=8.5 Hz, 1H, 6-Ph), 6.88 (s, 1H, 4-Th), 7.26 (s, 1H, 2-Th), 7.34 (s, 1H, 4-Ox), 7.59 (d, J=8.5 Hz, 1H, 5-Ph), 7.81 (s, 1H, 2-Ox).

### Example 16

### Synthesis of N-(5-phenylthien-3-ylmethyl)-3-methoxy-4-(oxazol-5-yl) aniline (16)

Use 3-methoxy-4-(oxazol-5-yl) aniline and 5-phenyl-thiophene-3-carbaldehyde as starting materials, follow the similar procedures applied in Example 1 to obtain compound 16.

¹H NMR (CDC₃, δ) 3.91 (s, 3H, —OCH₃), 4.56 (s, 2H, —CH₂—), 6.29 (s, 1H, 2-Ph), 6.38 (d, J=8.5 Hz, 1H, 6-Ph), 7.16 (s, 1H, 4-Th), 7.26 (s, 1H, 2-Th), 7.29 (t, J=7.5 Hz, J=7.5 Hz, 1H, 4′-Ph), 7.34 (s, 1H, 4-Ox), 7.37 (t, J=7.5 Hz, j=7.5 Hz, 2H, 3′, 5′-Ph), 7.55 (d, J=7.5 Hz, 2H, 2′, 6′-Ph), 7.58 (d, J=8.5 Hz, 1H, 5-Ph), 7.81 (s, 1H, 2-Ox).

### Example 17

### Synthesis of N-(1-methyl-1H-imidazol-5-yl methyl)-3-methoxy-4-(oxazol-5-yl) aniline (17)

Use 3-methoxy-4-(oxazol-5-yl) aniline and 1-methyl-1H-imidazol-5-yl ehanal as starting materials, follow the similar procedures applied in Example 1 to obtain compound 17.

¹H NMR (CDC₃, δ) 3.63 (s, 3H, —NCH3), 3.90 (s, 3H, —OCH₃), 4.27 (s, 2H, —CH₂—), 6.28 (s, 1H, 2-Ph), 6.35 (d, J=8.5 Hz, 1H, 6-Ph), 6.67 (s, 1H, 4-Imi), 7.19 (s, 1H, 2-Imi), 7.33 (s, 1H, 4-Ox), 7.58 (d, J=8.5 Hz, 1H, 5-Ph), 7.82 (s, 1H, 2-Ox).

### Example 18

### Synthesis of N-(5-methoxy-thien-3-yl methyl)-3-methoxy-4-(oxazol-5-yl) aniline (18)

Use 3-methoxy-4-(oxazol-5-yl) aniline and 5-methoxy-thiophene-3-carbaldehyde as starting materials, follow the similar procedures applied in Example 1 to obtain compound 18.

¹H NMR (CDC₃, δ) 3.89 (s, 3H, —OCH₃), 3.91 (s, 3H, CH₃O—Th), 4.48 (s, 2H, —CH₂—), 6.28 (s, 1H, 2-Ph), 6.38 (d, J=8.5 Hz, 1H, 6-Ph), 6.86 (s, 1H, 4-Th), 6.96 (s, 1H, 2-Th), 7.34 (s, 1H, 4-Ox), 7.59 (d, J=8.5 Hz, 1H, 5-Ph), 7.82 (s, 1H, 2-Ox).

### Example 19

### Synthesis of N-(5-methylthien-3-yl methylene)-3-methoxy-4-(oxazol-5-yl) aniline (19)

Use 3-methoxy-4-(oxazol-5-yl) aniline and 5-methylthiophene-3-carbaldehyde as starting materials, follow the similar procedures applied in Example 9 to obtain compound 19.

¹H NMR (CDC₃, δ) 2.45 (s, 3H, CH₃—Th), 3.99 (s, 3H, —OCH₃), 6.91 (s, 1H, 2-Ph), 6.92 (d, J=10.5 Hz, 1H, 6-Ph), 7.39 (s, 1H, 4-Th), 7.52 (s, 1H, 2-Th), 7.56 (s, 1H, 4-Ox), 7.78 (d, J=10.5 Hz, 1H, 5-Ph), 7.90 (s, 1H, 2-Ox), 8.62 (s, 1H, N═CH—).

### Example 20

### Synthesis of N-(benzothien-3-yl methyl)-3-methoxy-4-(oxazol-5-yl) aniline (20)

Use 3-methoxy-4-(oxazol-5-yl) aniline and benzothiophene-3-carbaldehyde as raw materials, follow the similar procedures applied in Example 1 to obtain compound 20, yield: 47.6%.

¹H NMR (CDC₃, δ) 3.88 (s, 3H, —OCH₃), 4.25 (br, 1H, NH), 4.61 (s, 2H, —CH₂), 6.28 (s, 1H, 2-Ph), 6.38 (d, J=8.5 Hz, 1H, 6-Ph), 7.35 (s, 1H, 4-Ox), 7.38 (s, 1H, 2-Th), 7.42 (m, 2H, 5,6-Th), 7.59 (d, J=8.5 Hz, 1H, 5-Ph), 7.82 (s, 1H, 2-Ox), 7.83 (m, 1H, 4-Th), 7.86 (m, 1H, 7-Th).

### Example 21

### Synthesis of N-(thien-2-ylmethyl)-2-methoxy-5-(oxazol-5-yl) aniline (21)

Use 2-methoxy-5-(oxazol-5-yl) aniline and thiophene-2-carbaldehyde as starting materials, follow the similar procedures applied in Example 1 to obtain compound 21, yield: 60.5%.

¹H NMR (CDC₃, δ) 3.88 (s, 3H, —OCH₃), 4.59 (s, 2H, —CH₂—), 4.85 (s, 1H, —NH), 6.81 (d, J=8.5 Hz, 1H, 3-Ph), 6.95 (s, 1H, 6-Ph), 6.99 (m, 1H, 3-Th), 7.06 (m, 1H, 4-Th), 7.16 (d, J=8.5 Hz, 1H, 4-Ph), 7.18 (s, 1H, 4-Ox), 7.23 (m, 1H, 5-Thj, 7.85 (s, 1H, 2-Ox).

### Example 22

### Synthesis of N-(5-methylthien-2-ylmethyl)-2-methoxy-5-(oxazol-5-yl) aniline (22)

Use 2-methoxy-5-(oxazol-5-yl) aniline and 5-methylthiophene-2-carbaldehyde as starting materials, follow the similar procedures applied in Example 1 to obtain compound 22, yield: 93.3%.

¹H NMR (CDC₃, δ) 2.46 (s, 3H, CH₃—Th), 3.87 (s, 3H, —OCH₃), 4.49 (s, 2H, —CH₂—), 4.74 (s, 1H, —NH), 6.61 (d, J=2 Hz, 3-Th), 6.80 (d, J=8.5 Hz, 1H, 3-Ph), 6.83 (d, J=3 Hz, 4-Th), 6.94 (s, 1H, 6-Ph), 7.18 (s, 1H, 4-Ox), 7.36 (d, J=8.5 Hz, 1H, 4-Ph), 7.82 (s, 1H, 2-Ox).

### Example 23

### Synthesis of N-(3,5-dimethylisoxazol-4-ylmethyl)-2-methoxy-5-(oxazol-5-yl) aniline (23)

Use 2-methoxy-5-(oxazol-5-yl) aniline and 3,5-dimethylisoxazole-4-carbaldehyde as raw materials, follow the similar procedures applied in Example 1 to obtain compound 23.

¹H NMR (CDC₃, δ) 2.35 (s, 6H, 3, 5-CH₃—Isox), 3.87 (s, 3H, —OCH₃), 4.48 (s, 2H, —CH₂—), 4.73 (s, 1H, —NH), 6.81 (d, J=8.5 Hz, 1H, 3-Ph), 6.93 (s, 1H, 6-Ph), 7.18 (s, 1H, 4-Ox), 7.35 (d, J=8.5 Hz, 1H, 4-Ph), 7.81 (s, 1H, 2-Ox).

### Example 24

### Synthesis of N-(5-chloromethylthien-2-ylmethyl)-2-methoxy-5-(oxazol-5-yl) aniline (24)

Use 2-methoxy-5-(oxazol-5-yl) aniline and 5-chloromethylthiophene-2-carbaldehyde as starting materials, follow the similar procedures applied in Example 1 to obtain compound 24.

¹H NMR (CDC₃, δ) 3.88 (s, 3H, —OCH₃), 4.47 (s, 2H, —CH₂—), 4.62 (s, 1H, —CH₂Cl), 6.61 (d, J=2 Hz, 3-Th), 6.81 (d, J=8.5 Hz, 1H, 3-Ph), 6.84 (d, J=2 Hz, 4-Th) 6.94 (s, 1H, 6-Ph), 7.17 (s, 1H, 4-Ox), 7.36 (d, J=8.5 Hz, 1H, 4-Ph), 7.82 (s, 1H, 2-Ox).

### Example 25

### Synthesis of N-(5-bromomethylthien-2-ylmethyl)-2-methoxy-5-(oxazol-5-yl) aniline (25)

Use 2-methoxy-5-(oxazol-5-yl) aniline and 5-bromomethylthiophene-2-carbaldehyde as starting materials, follow the similar procedures applied in Example 1 to obtain compound 25.

¹H NMR (CDC₃, δ) 3.89 (s, 3H, —OCH₃), 4.48 (s, 2H, —CH₂—), 4.64 (s, 2H, —CH₂Br), 6.62 (d, J=2 Hz, 3-Th), 6.82 (d, J=8.5 Hz, 1H, 3-Ph), 6.85 (d, J=2 Hz, 4-Th) 6.93 (s, 1H, 6-Ph), 7.18 (s, 1H, 4-Ox), 7.35 (d, J=8.5 Hz, 1H, 4-Ph), 7.81 (s, 1H, 2-Ox).

### Example 26

### Synthesis of N-(5-phenylthien-2-ylmethyl)-2-methoxy-5-(oxazol-5-yl) aniline (26)

Use 2-methoxy-5-(oxazol-5-yl) aniline and 5-phenylthiophene-2-carbaldehyde as starting materials, follow the similar procedures applied in Example 1 to obtain compound 26.

¹H NMR (CDC₃, δ) 3.88 (s, 3H, —OCH₃), 4.47 (s, 2H, —CH₂—), 6.83 (d, J=8.5 Hz, 1H, 3-Ph), 6.89 (d, J=2 Hz, 3-Th), 6.94 (s, 1H, 6-Ph), 7.05 (d, J=2 Hz, 4-Th), 7.18 (s, 1H, 4-Ox), 7.28 (t, J=7.5 Hz, J=7.5 Hz, 1H, 4′-Ph), 7.34 (d, J=8.5 Hz, 1H, 4-Ph), 7.38 (t, J=7.5 Hz, J=7.5 Hz, 2H, 3′, 5′-Ph), 7.54 (d, J=7.5 Hz, 2H, 2′, 6′-Ph), 7.82 (s, 1H, 2-Ox).

### Example 27

### Synthesis of N-(5-chloro-1,3-dimethyl-1H-pyrazol-4-yl methyl)-2-methoxy-5-(oxazol-5-yl) aniline (27)

Use 2-methoxy-5-(oxazol-5-yl) aniline and 1,3-dimethyl-5-chloropyrazole-4-carbaldehyde as starting materials, follow the similar procedures applied in Example 1 to obtain compound 27.

¹H NMR (CDC₃, δ) 2.78 (s, 3H, 3-CH₃—Pyra), 3.81 (s, 3H, N—CH₃—Pyra), 3.88 (s, 3H, —OCH₃), 4.47 (s, 2H, —CH₂—), 6.82 (d, J=8.5 Hz, 1H, 3-Ph), 6.94 (s, 1H, 6-Ph), 7.18 (s, 1H, 4-Ox), 7.36 (d, J=8.5 Hz, 1H, 4-Ph), 7.82 (s, 1H, 2-Ox).

### Example 28

### Synthesis of N-(2-phenyl-2H-1,2,3-triazol-4-ylmethyl) (2-methoxy-5-oxazole-5-yl) aniline (28)

Use 2-methoxy-5-(oxazol-5-yl) aniline and 2-phenyl-2H-[1,2,3]-triazole-4-carbaldehyde as starting materials, follow the similar procedures applied in Example 1 to obtain compound 28.

¹H NMR (CDC₃, δ) 3.89 (s, 3H, —OCH₃), 4.48 (s, 2H, —CH₂—), 6.84 (d, J=8.5 Hz, 1H, 3-Ph), 6.93 (s, 1H, 6-Ph), 7.19 (s, 1H, 4-Ox), 7.33 (m, 3H, 3, 4, 5-Ph′), 7.35 (d, J=8.5 Hz, 1H, 4-Ph), 7.81 (s, 1H, 2-Ox), 7.84 (s, 1H, 4-Tri), 8.12 (m, 2H, 2, 6-Ph′).

### Example 29

### Synthesis of N-(4-chlorothien-2-yl methylene)-2-methoxy-5-(oxazol-5-yl)-aniline (29)

Use 2-methoxy-5-(oxazol-5-yl) aniline and 4-chlorothiophene-2-carbaldehyde as starting materials, follow the similar procedures applied in Example 9 to obtain compound 29.

¹H NMR (CDC₃, δ) 3.99 (s, 3H, —OCH₃), 6.92 (s, 1H, 3-Th), 7.09 (s, 1H, 5-Th), 7.36 (d, J=8.5 Hz, 1H, 4-Ph), 7.44 (s, 1H, 6-Ph), 7.49 (d, J=8.5 Hz, 1H, 3-Ph), 7.38 (s, 1H, 4-Ox), 7.90 (s, 1H, 2-Ox), 8.13 (s, 1H, N═CH—).

### Example 30

### Synthesis of N-(5-chlorobenzothien-2-ylmethyl)-2-methoxy-5-(oxazol-5-yl) aniline (30)

Use 2-methoxy-5-(oxazol-5-yl) aniline and 5-chlorobenzothiophene-2-carbaldehyde as raw materials, follow the similar procedures applied in Example 1 to obtain compound 30.

¹H NMR (CDC₃, δ) 3.87 (s, 3H, —OCH₃), 4.67 (s, 2H, —CH₂—), 6.84 (d, J=8.5 Hz, 1H, 3-Ph), 6.98 (s, 1H, 6-Ph), 7.18 (s, 1H, 4-Ox), 7.26 (s, 1H, 3-Th), 7.33 (m, 1H, 6-Th), 7.40 (d, J=8.5 Hz, 1H, 4-Ph), 7.71 (s, 1H, 4-Th), 7.82 (m, 1H, 7-Th), 7.82 (s, 1H, 2-Ox).

### Example 31

### Synthesis of N-(thien-3-yl methyl)-2-methoxy-5-(oxazol-5-yl) aniline (31)

Use 2-methoxy-5-(oxazol-5-yl) aniline and thiophene-3-carbaldehyde as starting materials, follow the similar procedures applied in Example 1 to obtain compound 31.

¹H NMR (CDC₃, δ) 3.88 (s, 3H, —OCH₃), 4.53 (s, 2H, —CH₂—), 6.82 (d, J=8.5 Hz, 1H, 3-Ph), 6.94 (s, 1H, 6-Ph), 7.08 (d, J=5 Hz, 1H, 4-Th), 7.15 (d, J=8.5 Hz, 1H, 4-Ph), 7.18 (s, 1H, 4-Ox), 7.21 (s, 1H, 2-Th), 7.33 (d, J=5 Hz, 1H, 5-Th), 7.84 (s, 1H, 2-Ox).

### Example 32

### Synthesis of N-(5-methylthien-3-yl methyl)-N-(2-methoxy-5-oxazol-5-yl) acrylamide (32)

Use 2-methoxy-5-(oxazol-5-yl) aniline (1 mmol) and 5-methyl-thiophene-3-carbaldehyde (1 mmol) as the starting materials, follow the similar procedures applied in Example 1 to isolate the product N-(5-methyl-thiophene-3-methyl)-2-methoxy-5-(oxazol-5-yl) aniline, dissolve it in tetrahydrofuran, add slowly propionyl chloride dropwise until the disappearance of the intermediate. Separate and purify the product to obtain compound 32.

¹H NMR (CDC₃, δ) 1.13 (t, 3H, COCH₂CH₃), 2.27 (q, 2H, COCH₂CH₃), 2.43 (s, 3H, CH₃—Th), 3.87 (s, 3H, —OCH₃), 4.65 (s, 2H, —CH₂—), 6.87 (d, J=8.5 Hz, 1H, 3-Ph), 6.87 (s, 1H, 4-Th), 7.21 (s, 1H, 6-Ph), 7.34 (d, J=8.5 Hz, 1H, 4-Ph), 7.18 (s, 1H, 4-Ox), 7.20 (s, 1H, 2-Th), 7.82 (s, 1H, 2-Ox).

### Example 33

### Synthesis of N-(2-methoxy-5-oxazol-5-yl phenyl)-1-methylpiperidin-4-yl methylamine (33)

Use 2-methoxy-5-(oxazol-5-yl) aniline and 1-methyl-piperidine-4-carbaldehyde as starting materials, follow the similar procedures applied in Example 1 to obtain compound 33.

¹H NMR (CDC₃, δ) 1.46 (m, 4H, 3, 5-Pip), 1.73 (m, 1H, 4-Pip), 2.24 (m, 4H, 2, 6-Pip), 2.26 (s, 3H, N—CH₃—Pip), 3.02 (m, 2H, —CH₂—), 3.88 (s, 3H, —OCH₃), 6.82 (d, J=8.5 Hz, 1H, 3-Ph), 6.94 (s, 1H, 6-Ph), 7.15 (d, J=8.5 Hz, 1H, 4-Ph), 7.18 (s, 1H, 4-Ox), 7.81 (s, 1H, 2-Ox).

### Example 34

### Synthesis of N-(5-chlorothien-3-yl methyl)-2-methoxy-5-(oxazol-5-yl) aniline (34)

Use 2-methoxy-5-(oxazol-5-yl) aniline and 5-chlorothiophene-3-carbaldehyde as starting materials, follow the similar procedures applied in Example 1 to obtain compound 34.

¹H NMR (CDC₃, δ) 3.87 (s, 3H, —OCH₃), 4.48 (s, 2H, —CH₂—), 6.80 (d, J=8.5 Hz, 1H, 3-Ph), 6.89 (s, 1H, 4-Th), 6.92 (s, 1H, 6-Ph), 7.16 (d, J=8.5 Hz, 1H, 4-Ph), 7.18 (s, 1H, 4-Ox), 7.24 (s, 1H, 2-Th), 7.81 (s, 1H, 2-Ox).

### Example 35

### Synthesis of N-(5-bromothien-3-yl methyl)-2-methoxy-5-(oxazol-5-yl) aniline (35)

Use 2-methoxy-5-(oxazol-5-yl) aniline and 5-bromo-thiophene-3-carbaldehyde as starting materials, follow the similar procedures applied in Example 1 to obtain compound 35.

¹H NMR (CDC₃, δ) 3.87 (s, 3H, —OCH₃), 4.48 (s, 2H, —CH₂—), 6.80 (d, J=8.5 Hz, 1H, 3-Ph), 6.92 (s, 1H, 6-Ph), 7.07 (s, 1H, 4-Th), 7.16 (d, J=8.5 Hz, 1H, 4-Ph), 7.18 (s, 1H, 4-Ox), 7.25 (s, 1H, 2-Th), 7.81 (s, 1H, 2-Ox).

### Example 36

### Synthesis of N-(5-phenylthien-3-yl methyl)-2-methoxy-5-(oxazol-5-yl) aniline (36)

Use 2-methoxy-5-(oxazol-5-yl) aniline and 5-phenyl-thiophene-3-carbaldehyde as starting materials, follow the similar procedures applied in Example 1 to obtain compound 36.

¹H NMR (CDC₃, δ) 3.89 (s, 3H, —OCH₃), 4.48 (s, 2H, —CH₂—), 6.83 (d, J=8.5 Hz, 1H, 3-Ph), 6.88 (s, 1H, 4-Th), 6.95 (s, 1H, 6-Ph), 7.16 (d, J=8.5 Hz, 1H, 4-Ph), 7.18 (s, 1H, 4-Ox), 7.20 (s, 1H, 2-Th), 7.24 (m, 1H, 4′-Ph), 7.32 (n, 2H, 3′, 5′-Ph), 7.48 (m, 2H, 2′, 6′-Ph), 7.82 (s, 1H, 2-Ox).

### Example 37

### Synthesis of N-(tetrahydropyran-4-yl methyl)-2-methoxy-5-oxazol-5-yl aniline (37)

Use 2-methoxy-5-(oxazol-5-yl) aniline and tetrahydropyran-4-carbaldehyde as starting materials, follow the similar procedures applied in Example 1 to obtain compound 37.

¹H NMR (CDC₃, δ) 1.56 (m, 4H, 3, 5-Pyr), 1.83 (m, 1H, 4-Pyr), 3.04 (m, 2H, —CH₂—), 3.59 (m, 4H, 2, 6-Pyr), 3.87 (s, 3H, —OCH₃), 6.83 (d, J=8.5 Hz, 1H, 3-Ph), 6.93 (s, 1H, 6-Ph), 7.16 (d, J=8.5 Hz, 1H, 4-Ph), 7.19 (s, 1H, 4-Ox), 7.82 (s, 1H, 2-Ox).

### Example 38

### Synthesis of N-(pyridazin-3-yl methyl)-2-methoxy-5-oxazol-5-yl aniline (38)

Use 2-methoxy-5-(oxazol-5-yl) aniline and pyridazine-3-carbaldehyde as starting materials, follow the similar procedures applied in Example 1 to obtain compound 38.

¹H NMR (CDC₃, δ) 3.89 (s, 3H, —OCH₃), 4.48 (s, 2H, —CH₂—), 6.82 (d, J=8.5 Hz, 1H, 3-Ph), 6.94 (s, 1H, 6-Ph), 7.14 (d, J=8.5 Hz, 1H, 4-Ph), 7.18 (s, 1H, 4-Ox), 7.58 (n, 2H, 4, 5-Pyr), 7.82 (s, 1H, 2-Ox), 8.87 (n, 1H, 6-Pyr).

### Example 39

### Synthesis of N-(thien-3-yl methylene)-2-methoxy-5-(oxazol-5-yl) aniline (39)

Use 2-methoxy-5-(oxazol-5-yl) aniline and thiophene-3-carbaldehyde as starting materials, follow the similar procedures applied in Example 9 to obtain compound 39.

¹H NMR (CDC₃, δ) 3.98 (s, 3H, —OCH₃), 6.96 (d, J=4 Hz, 1H, 4-Th), 7.10 (d, J=4 Hz, 1H, 5-Th), 7.20 (s, 1H, 3-Th), 7.32 (d, J=8.5 Hz, 1H, 4-Ph), 7.43 (s, 1H, 6-Ph), 7.44 (d, J=8.5 Hz, 1H, 3-Ph), 7.35 (s, 1H, 4-Ox), 7.88 (s, 1H, 2-Ox), 8.13 (s, 1H, N═CH—).

### Example 40

### Synthesis of N-(6-bromo-7-methyl benzothien-3-yl methyl)-2-methoxy-5-oxazol-5-yl aniline (40)

Use 2-methoxy-5-(oxazol-5-yl) aniline and 6-bromo-7-methyl-benzothiophene-3-carbaldehyde as starting materials, follow the similar procedures applied in Example 1 to obtain compound 40.

¹H NMR (CDC₃, δ) 2.35 (s, 3H, CH₃—Th), 3.88 (s, 3H, —OCH₃), 4.48 (s, 2H, —CH₂—), 6.82 (d, J=8.5 Hz, 1H, 3-Ph), 6.95 (s, 1H, 6-Ph), 7.06 (d, J=8 Hz, 1H, 5-Th), 7.16 (d, J=8.5 Hz, 1H, 4-Ph), 7.18 (s, 1H, 4-Ox), 7.29 (d, J=8 Hz, 1H, 4-Th), 7.38 (s, 1H, 2-Th), 7.82 (s, 1H, 2-Ox).

### Example 41

### Synthesis of N-(furan-2-yl methyl)-3-methoxy-4-(oxazol-5-yl) aniline (41)

Use 3-methoxy-4-(oxazol-5-yl) aniline and 2-furaldehyde as starting materials, follow the similar procedures applied in Example 1 to obtain compound 41, yield: 51.8%.

¹H NMR (CDC₃, δ) 3.90 (s, 3H, —OCH₃), 4.37 (s, 2H, —CH₂—), 6.26 (d, 1H, 3-Fu), 6.27 (s, 1H, 2-Ph), 6.36 (d, J=8.5 Hz, 1H, 6-Ph), 6.34 (n, 1H, 4-Fu), 7.34 (s, 1H, 4-Ox), 7.38 (n, 1H, 5-Fu), 7.57 (d, 1H, J=8.5 Hz, 5-Ph), 7.81 (s, 1H, 2-Ox).

### Example 42

### Synthesis of N-(5-methylfuran-2-yl methyl)-3-methoxy-4-(oxazol-5-yl) aniline (42)

Use 3-methoxy-4-(oxazol-5-yl) aniline and 5-methyl-2-furaldehyde as raw materials, follow the similar procedures applied in Example 1 to obtain compound 42, yield: 68.0%.

¹H NMR (CDC₃, δ) 2.28 (s, 3H, CH₃-Fu), 3.90 (s, 3H, —OCH₃), 4.30 (s, 2H, —CH₂), 5.91 (d, J=2 Hz, 1H, 4-Fu), 6.13 (d, J=2.5 Hz, 1H, 3-Fu), 6.28 (s, 1H, 2-Ph), 6.35 (d, J=8.5 Hz, 1H, 6-Ph), 7.33 (s, 1H, 4-Ox), 7.57 (d, J=8.5 Hz, 1H, 5-Ph), 7.81 (s, 1H, 2-Ox).

### Example 43

### Synthesis of N-(2,4,6-trichloropyrimidin-5-yl methyl)-3-methoxy-4-(oxazol-5-yl) aniline (43)

Use 3-methoxy-4-(oxazol-5-yl) aniline and 2,4,6-trichloro-pyrimidine-5-carbaldehyde as starting materials, follow the similar procedures applied in Example 1 to obtain compound 43.

¹H NMR (CDC₃, δ) 3.89 (s, 3H, —OCH₃), 4.31 (s, 2H, —CH₂), 6.29 (s, 1H, 2-Ph), 6.34 (d, J=8.5 Hz, 1H, 6-Ph), 7.34 (s, 1H, 4-Ox), 7.58 (d, J=8.5 Hz, 1H, 5-Ph), 7.82 (s, 1H, 2-Ox).

### Example 44

### Synthesis of N-(3,5-dichloropyrazin-2-yl methyl)-3-methoxy-4-(oxazol-5-yl) aniline (44)

Use 3-methoxy-4-(oxazol-5-yl) aniline and 3,5-dichloropyrazine-2-yl methyl as raw materials, follow the similar procedures applied in Example 1 to obtain compound 44.

¹H NMR (CDC₃, δ) 3.89 (s, 3H, —OCH₃), 4.30 (s, 2H, —CH₂), 6.29 (s, 1H, 2-Ph), 6.35 (d, J=8.5 Hz, 1H, 6-Ph), 7.34 (s, 1H, 4-Ox), 7.55 (d, J=8.5 Hz, 1H, 5-Ph), 7.82 (s, 1H, 2-Ox), 8.50 (s, 1H, 6-Pyra).

### Example 45

### Synthesis of N-(5-bromofuran-2-yl methyl)-2-methoxy-5-(oxazol-5-yl) aniline (45)

Use 2-methoxy-5-(oxazol-5-yl) aniline and 5-bromo-furaldehyde-2 as starting materials, follow the similar procedures applied in Example 1 to obtain compound 45.

¹H NMR (CDC₃, δ) 3.89 (s, 3H, —OCH₃), 4.48 (s, 2H, —CH₂—), 6.45 (d, J=3 Hz, 3-Fu), 6.70 (d, J=3 Hz, 1H, 4-Fu), 6.80 (d, J=8.5 Hz, 1H, 3-Ph), 6.95 (s, 1H, 6-Ph), 7.18 (s, 1H, 4-Ox), 7.37 (d, J=8.5 Hz, 1H, 4-Ph), 7.82 (s, 1H, 2-Ox).

### Example 46

### Synthesis of N-[5-(3-chlorophenyl)-furan-2-yl methyl-2-methoxy-5-(oxazol-5-yl) aniline (46)

Use 2-methoxy-5-(oxazol-5-yl) aniline and 5-(3-chlorophenyl)-furaldehyde-2 as starting materials, follow the similar procedures applied in Example 1 to obtain compound 46.

¹H NMR (CDC₃, δ) 3.88 (s, 3H, —OCH₃), 4.47 (s, 2H, —CH₂—), 6.35 (d, J=3 Hz, 3-Fu), 6.70 (d, J=3 Hz, 1H, 4-Fu), 6.82 (d, J=8.5 Hz, 1H, 3-Ph), 6.95 (s, 1H, 6-Ph), 7.18 (s, 1H, 4-Ox), 7.22 (d, J=8 Hz, 1H, 4′-Ph), 7.25 (dd, J=8 Hz, 8 Hz, 1H, 5′-Ph), 7.35 (d, J=8.5 Hz, 1H, 4-Ph), 7.37 (d, J=8 Hz, 1H, 6′-Ph), 7.48 (s, 1H, 2′-Ph), 7.81 (s, 1H, 2-Ox).

### Example 47

### Synthesis of N-2-(3,4,5-trimethoxyphenylaminocarbonyl) furan-5-yl methyl-3-methoxy-4-(oxazol-5-yl) aniline (47)

Use 3-methoxy-4-(oxazol-5-yl) aniline and 5-formyl-2-furic acid as raw materials, follow the similar procedures applied in Example 7 to obtain the compound 5-(3-methyl-4-oxazol-5-yl-anilino)-methyl-2-furic acid, which is subsequently condensated with 3,4,5-trimethoxy aniline in the presence of DIC and HOBt, after flash separation and purification, to obtain product 47.

¹H NMR (CDC₃, δ) 3.90 (s, 3H, —OCH₃), 3.96 (s, 9H, 3′, 4′, 5′-OCH₃), 4.32 (s, 2H, —CH₂), 6.28 (s, 1H, 2-Ph), 6.35 (d, J=8.5 Hz, 1H, 6-Ph), 6.37 (d, J=3 Hz, 1H, 4-Fu), 6.61 (s, 2H, 26′-Ph), 7.13 (d, J=3 Hz, 1H, 3-Fu), 7.33 (s, 1H, 4-Ox), 7.57 (d, J=8.5 Hz, 1H, 5-Ph), 7.81 (s, 1H, 2-Ox), 13.2 (s, 1H, —CONH).

### Example 48

### Synthesis of N-(5-methoxymethylfuran-2-yl methyl)-2-methoxy-5-(oxazol-5-yl) aniline (48)

Use 2-methoxy-5-(oxazol-5-yl) aniline and 5-methoxymethyl-furaldehyde-2 as raw materials, follow the similar procedures applied in Example 1 to obtain compound 48.

¹H NMR (CDC₃, δ) 3.24 (s, 3H, —OCH₃-Fu), 3.89 (s, 3H, —OCH₃), 4.15 (s, 2H, —CH₂—), 4.48 (s, 2H, —CH₂—), 6.45 (d, J=3 Hz, 3-Fu), 6.70 (d, J=3 Hz, 1H, 4-Fu), 6.80 (d, J=8.5 Hz, 1H, 3-Ph), 6.95 (s, 1H, 6-Ph), 7.18 (s, 1H, 4-Ox), 7.37 (d, J=8.5 Hz, 1H, 4-Ph), 7.82 (s, 1H, 2-Ox).

### Example 49

### Synthesis of N-(5-nitrofuran-2-methylidene)-3-methoxy-4-(oxazol-5-yl) aniline (49)

Use 3-methoxy-4-(oxazol-5-yl) aniline and 5-nitro-furaldehyde-2 as starting materials, follow the similar procedures applied in Example 9 to obtain compound 49, yield: 30.5%.

¹H N NMR (CDC₃, δ) 4.00 (s, 3H, —OCH₃), 6.96 (s, 1H, 2-Ph), 6.98 (d, J=8 Hz, 1H, 6-Ph), 7.23 (d, J=4 Hz, 1H, 3-Fu), 7.44 (d, J=3.5 Hz, 1H, 4-Fu), 7.60 (s, 1H, 4-Ox), 7.83 (d, J=8 Hz, 1H, 5-Ph), 7.93 (s, 1H, 2-Ox), 8.47 (s, 1H, N═CH—).

### Example 50

### Synthesis of N-(benzofuran-3-ylmethyl)-2-methoxy-5-(oxazol-5-yl) aniline (50)

Use 2-methoxy-5-(oxazol-5-yl) aniline and benzofuraldehyde-3 as starting materials, follow the similar procedures applied in Example 1 to obtain compound 50.

¹H NMR (CDC₃, δ) 3.88 (s, 3H, —OCH₃), 4.47 (s, 2H, —CH₂—), 6.81 (d, J=8.5 Hz, 1H, 3-Ph), 6.95 (s, 1H, 6-Ph), 7.15 (m, 2H, 5, 6-Fu), 7.18 (s, 1H, 4-Ox), 7.24 (s, 1H, 2-Fu), 7.36 (d, J=8.5 Hz, 1H, 4-Ph), 7.45 (m, 2H, 4, 7-Fu), 7.82 (s, 1H, 2-Ox).

### Example 51

### Synthesis of N-(1-methyl-1H-pyrrol-2-yl methyl)-3-methoxy-4-(oxazol-5-yl) aniline (51)

Use 3-methoxy-4-(oxazol-5-yl) aniline and N-methylpyrrole aldehyde-2 as the raw materials, follow the similar procedures applied in Example 1 to obtain compound 51, yield: 14.1%.

¹H NMR (CDC₃, δ) 3.48 (s, 3H, N—CH₃), 3.88 (s, 3H, —OCH₃), 4.36 (s, 2H, —CH₂), 5.59 (m, 1H, 3-Pyr), 5.77 (m, 1H, 4-Pyr), 6.12 (m, 1H, 5-Pyr), 6.32 (s, 1H, 2-Ph), 6.36 (d, J=8.5 Hz, 1H, 6-Ph), 7.34 (s, 1H, 4-Ox), 7.56 (d, J=8.5 Hz, 1H, 5-Ph), 7.81 (s, 1H, 2-Ox).

### Example 52

### Synthesis of N-(5-methoxy-1H-indol-3-yl methyl)-3-methoxy-4-(oxazol-5-yl) aniline (52)

Use 3-methoxy-4-(oxazol-5-yl) aniline and 5-methoxy-1H-indole-3-carbaldehyde as raw materials, follow the similar procedures applied in Example 1 to obtain compound 52, yield 28.6%.

¹H NMR (CDC₃, δ) 3.89 (s, 3H, —OCH₃), 3.94 (s, 3H, CH₃O-Ind), 4.02 (br, 1H, —NH), 4.20 (s, 2H, —CH₂), 6.31 (s, 1H, 2-Ph), 6.35 (d, J=8.5 Hz, 1H, 6-Ph), 6.69 (m, 1H, 6-Ind), 6.86 (s, 1H, 2-Ind), 7.07 (m, 1H, 7-Ind), 7.33 (s, 1H, 4-Ox), 7.40 (m, 1H, 4-Ind), 7.57 (d, J=8.5 Hz, 1H, 5-Ph), 7.82 (s, 1H, 2-Ox), 8.05 (br, 1H, —NH-Ind).

### Example 53

### Synthesis of N-(1H-indol-3-yl methyl)-3-methoxy-4-(oxazol-5-yl) aniline (53)

Use 3-methoxy-4-(oxazol-5-yl) aniline and indole-3-carbaldehyde as starting materials, follow the similar procedures applied in Example 1 to obtain compound 53, yield: 16.7%.

¹H NMR (CDC₃, δ) 3.89 (s, 3H, —OCH₃), 4.02 (br, 1H, NH), 4.20 (s, 2H, —CH₂), 6.30 (s, 1H, 2-Ph), 6.36 (d, J=8.5 Hz, 1H, 6-Ph), 6.87 (s, 1H, 2-Ind), 7.07 (m, 1H, 7-Ind), 7.11 (m, 1H, 6-Ind), 7.19 (m, 1H, 5-Ind), 7.32 (s, 1H, 4-Ox), 7.40 (m, 1H, 4-Ind), 7.56 (d, J=8.5 Hz, 1H, 5-Ph), 7.81 (s, 1H, 2-Ox), 8.05 (br, 1H, —NH-Ind).

### Example 54

### Synthesis of N-(4-methylthiazol-5-yl methyl)-3-methoxy-4-(oxazol-5-yl) aniline (54)

Use 3-methoxy-4-(oxazol-5-yl) aniline and 4-methyl-thiazole-5-carbaldehyde as starting materials, follow the similar procedures applied in Example 1 to obtain compound 54, yield: 75.7%.

¹H NMR (CDC₃, δ) 2.51 (s, 3H, CH₃-Thia), 3.89 (s, 3H, —OCH₃), 4.50 (s, 2H, —CH₂), 6.24 (s, 1H, 2-Ph), 6.33 (d, J=8.5 Hz, 1H, 6-Ph), 7.35 (s, 1H, 4-Ox), 7.59 (d, J=8.5 Hz, 1H, 5-Ph), 7.82 (s, 1H, 2-Ox), 8.64 (s, 1H, 2-Thia).

### Example 55

### Synthesis of N-N-(thiazol-5-yl methyl)-3-methoxy-4-(oxazol-5-yl) aniline (55)

Use 3-methoxy-4-(oxazol-5-yl) aniline and thiazole-5-carbaldehyde as raw materials, follow the similar procedures applied in Example 1 to obtain compound 55, yield: 83.5%.

¹H NMR (CDC₃, δ) 3.89 (s, 3H, —OCH₃), 4.64 (s, 2H, —CH₂), 6.27 (s, 1H, 2-Ph), 6.36 (d, J=8.5 Hz, 1H, 6-Ph), 7.35 (s, 1H, 4-Ox), 7.59 (d, J=8 Hz, 1H, 5-Ph), 7.83 (s, 1H, 2-Ox), 7.85 (s, 1H, 4-Thia), 8.75 (s, 1H, 2-Thia).

### Example 56

### Synthesis of N-(pyridin-4-yl methyl)-3-methoxy-4-(oxazol-5-yl) aniline (56)

Use 3-methoxy-4-(oxazol-5-yl) aniline and 4-pyridylaldehyde as the starting materials, follow the similar procedures applied in Example 1 to obtain compound 56, yield: 46.2%.

¹H NMR (CDC₃, δ) 3.84 (s, 3H, —OCH₃), 4.43 (s, 2H, —CH₂—), 6.18 (s, 1H, 2-Ph), 6.25 (d, J=8.5 Hz, 1H, 6-Ph), 7.30 (d, J=7.5 Hz, 2H, 3, 5-Py), 7.33 (s, 1H, 4-Ox), 7.55 (d, J=8.5 Hz, 1H, 5-Ph), 7.81 (s, 1H, 2-Ox), 8.57 (d, J=7.5 Hz, 2H, 2, 6-Py).

### Example 57

### Synthesis of N-(6-methylpyridin-2-yl methyl)-3-methoxy-4-(oxazol-5-yl) aniline (57)

Use 3-methoxy-4-(oxazol-5-yl) aniline and 6-methylpyridyl aldehyde-2 as starting materials, follow the similar procedures applied in Example 1 to obtain compound 57, yield: 73.8%.

¹H NMR (CDC₃, δ) 2.59 (s, 3H, CH₃—Py), 3.90 (s, 3H, —OCH₃), 4.46 (s, 2H, —CH₂), 5.10 (br, 1H, NH), 6.29 (s, 1H, 2-Ph), 6.35 (d, J=8.5 Hz, 1H, 6-Ph), 7.07 (d, J=7.5 Hz, 1H, 5-Py), 7.14 (d, J=8 Hz 1H, 3-Py), 7.32 (s, 1H, 4-Ox), 7.56 (t, J=7.5 Hz, J=8 Hz 1H, 4-Py), 7.57 (d, J=8.5 Hz, 1H, 5-Ph), 7.81 (s, 1H, 2-Ox).

### Example 58

### Synthesis of 2-methoxy-4-(oxazol-5-yl)-5-[(quinolin-4-yl methyl)-amino]phenol (58)

Use 5-amino-5-methoxy-4-oxazol-4-yl phenol and quinoline carbaldehyde as raw materials, follow the similar procedures applied in Example 1 to obtain compound 58, yield: 45.8%.

¹H NMR (CDC₃, δ) 3.85 (s, 3H, —OCH₃), 4.11 (br, 1H, NH), 4.42 (s, 2H, —CH₂—), 5.01 (br, 1H, OH), 7.03 (d, J=8 Hz, 1H, 3-Qui), 7.34 (s, 1H, 4-Ox), 7.41 (m, 1H, 6-Qui), 7.57 (m, 1H, 7-Qui), 7.77 (m, 1H, 5-Qui), 7.82 (s, 1H, 2-Ox), 8.02 (m, 1H, 8-Qui), 8.67 (d, J=8 Hz, 1H, 2-Qui).

### Example 59

### Synthesis of N-(2-chloropyridin-3-yl methyl)-3-methoxy-4-(oxazol-5-yl) aniline (59)

Use 3-methoxy-4-(oxazol-5-yl) aniline and 2-chloro-3-pyridyl aldehyde as the starting materials, follow the similar procedures applied in Example 1 to obtain compound 59, yield: 56.2%.

¹H NMR (CDC₃, 5) 3.86 (s, 3H, —OCH₃), 4.50 (s, 2H, —CH₂), 6.19 (s, 1H, 2-Ph), 6.25 (d, J=8.5 Hz, 1H, 6-Ph), 7.22 (m, 1H, 5-Py), 7.34 (s, 1H, 4-Ox), 7.56 (d, J=8.5 Hz, 1H, 5-Ph), 7.74 (1H, m, 4-Py), 7.82 (s, 1H, 2-Ox), 8.32 (m, 1H, 6-Py).

### Example 60

### Synthesis of N-(6-methoxy-pyridin-3-yl methyl)-3-methoxy-4-(oxazol-5-yl) aniline (60)

Use 3-methoxy-4-(oxazol-5-yl) aniline and 2-methoxy-5-pyridyl aldehyde as starting materials, follow the similar procedures applied in Example 1 to obtain compound 60, yield: 51.7%.

¹H NMR (CDC₃, δ) 3.88 (s, 3H, —OCH₃), 3.94 (s, 3H, CH₃O-Py), 4.31 (s, 2H, —CH₂), 6.23 (s, 1H, 2-Ph), 6.33 (d, J=8.5 Hz, 1H, 6-Ph), 6.75 (d, J=8.5 Hz, 1H, 3-Py), 7.33 (s, 1H, 4-Ox), 7.57 (d, J=8.5 Hz, 1H, 5-Ph), 7.60 (d, J=8.5 Hz, 1H, 4-Py), 7.82 (s, 1H, 2-Ox), 8.17 (m, 1H, 6-Py).

### Example 61

### Synthesis of N-(4-methoxybenzyl)-3-methoxy-4-(oxazol-5-yl) aniline (61)

Use 3-methoxy-4-(oxazol-5-yl) aniline and p-methoxybenzaldehyde as raw materials, follow the similar procedures applied in Example 1 to obtain compound 61, yield: 67.7%.

¹H NMR (CDC₃, δ) 3.81 (s, 3H, CH3O-Ph′), 3.87 (s, 3H, OCH₃), 4.31 (s, 2H, —CH₂—), 6.22 (s, 1H, 2-Ph), 6.32 (d, J=8.5 Hz, 1H, 6-Ph), 6.89 (d, J=8.5 Hz, 2H, 2′, 6′-Ph,), 7.30 (d, J=8.5 Hz, 2H, 3′, 5′-Ph), 7.32 (s, 1H, 4-Ox), 7.56 (d, J=8.5 Hz, 1H, 5-Ph), 7.81 (s, 1H, 2-Ox).

### Example 62

### Synthesis of N-(3-phenylpropen-2-yl)-3-methoxy-4-(oxazol-5-yl) aniline (62)

Use 3-methoxy-4-(oxazol-5-yl) aniline and trans-cinnamaldehyde as the raw materials, follow the similar procedures applied in Example 1 to obtain compound 62, yield: 15.7%.

¹H NMR (CDC₃, δ) 3.91 (s, 3H, —OCH₃), 4.00 (d, J=5 Hz, 1H, —CH₂—), 6.31 (s, 1H, 2-Ph), 6.33 (n, 1H, —CH₂—CH═CH—), 6.39 (d, J=8.5 Hz, 1H, 6-Ph), 6.65 (d, J=16 Hz, 1H, —CH₂—CH═CH—), 7.21 (m, 1H, 4′-Ph), 731 (t, J=7.5 Hz, J=7.5 Hz, 2H, 3′, 5′-Ph), 7.37 (d, J=7 Hz, 2H, 2′, 6′-Ph), 7.34 (s, 1H, 4-Ox), 7.58 (d, J=8.5 Hz, 1H, 5-Ph), 7.82 (s, 1H, 2-Ox).

### Example 63

### Synthesis of N-[3-(2-methoxyphenylpropen-2-yl]-3-methoxy-4-(oxazol-5-yl) aniline (63)

Use 3-methoxy-4-(oxazol-5-yl) aniline and o-methoxy cinnamaldehyde as the raw materials, follow the similar procedures applied in Example 1 to obtain compound 63, yield: 77.4%.

¹H NMR (CDC₃, δ) 3.85 (s, 3H, 2′-OCH₃), 3.91 (s, 3H, —OCH₃), 4.00 (d, J=6 Hz, 1H, —CH₂—), 6.30 (s, 1H, 2-Ph), 6.33 (dd, J=6 Hz, J=16 Hz, 1H, —CH₂—CH═CH—), 6.37 (d, J=8.5 Hz, 1H, 6-Ph), 6.88 (d, J=8 Hz, 1H, 3′-Ph), 6.92 (t, J=8 Hz, J=8 Hz, 1H, 5′-Ph), 6.97 (d, J=16 Hz, 1H, —CH₂—CH═CH—), 7.23 (t, J=8 Hz, J=8 Hz, 1H, 4′-Ph), 7.33 (s, 1H, 4-Ox), 7.42 (d, J=8 Hz, 1H, 6′-Ph), 7.57 (d, J=8.5 Hz, 1H, 5-Ph), 7.81 (s, 1H, 2-Ox).

### Example 64

### Synthesis of N-(4-Fluorophenylmethylene)-3-methoxy-4-(oxazol-5-yl)-aniline (64)

Use 3-methoxy-4-(oxazol-5-yl) aniline and p-fluorobenzaldehyde as starting materials, follow the similar procedures applied in Example 9 to obtain compound 64, yield: 26.9%.

¹H NMR (CDC₃, δ) 4.01 (s, 3H, —OCH₃), 6.88 (s, 1H, 2-Ph), 6.98 (d, J=10.5 Hz, 1H, 6-Ph), 7.19 (m, 2H, 2′, 6′-Ph), 7.92 (m, 2H, 3′, 5′-Ph), 7.58 (s, 1H, 4-Ox), 7.80 (d, J=11 Hz, 1H, 5-Ph), 7.91 (s, 1H, 2-Ox), 8.48 (s, 1H, N═CH—).

### Example 65

### Synthesis of N-(phenylpropen-1-ylidene)-3-methoxy-4-(oxazol-5-yl)-aniline (65)

Use 3-methoxy-4-(oxazol-5-yl) aniline and trans-cinnamaldehyde as the raw materials, follow the similar procedures applied in Example 9 to obtain compound 65 as a yellow solid, yield: 23.0%.

¹H NMR (CDC₃, δ) 4.01 (s, 3H, —OCH₃), 6.88 (s, 1H, 2-Ph), 6.89 (d, J=8.5 Hz, 1H, 6-Ph), 7.21 (m, 1H, 4′-Ph) 7.39 (m, 2H, 3′, 5′-Ph), 7.44 (m, 2H, 2′, 6′-Ph), 7.55 (m, 1H, C═CH-Ph), 7.56 (s, 1H, 4-Ox), 7.57 (m, 1H, 5-Ph), 7.79 (d, J=10.5 Hz, 1H, N═CH—CH), 7.91 (s, 1H, 2-Ox), 8.35 (d, J=10.5 Hz, 1H, N═CH—).

### Example 66

### Synthesis of (4-Methoxy-2-methylphenyl) hydrazonomalononitrile (66)

Dissolve 3-methoxy-4-(oxazol-5-yl) aniline (190.2 mg, 1 mmol) in dilute 0.75 N HCl (8.5 ml) in a 25 ml flask cooled in ice-bath, add in NaNO₂ (1.5 mmol) solution dropwise, subsequently stir for 10 minutes, then add in malononitrile (1.5 mmol), stir for 2 h at room temperature, extract the reactant with dichloromethane. Treat the extractant conventionally to obtain product 66 as a tan solid, yield: 70.5%.

¹H NMR (CDC₃, δ) 4.04 (s, 3H, —OCH₃), 6.91 (m, 1H, 6-Ph), 7.03 (s, 1H, 2-Ph), 7.82 (d, J=10 Hz, 1H, 5-Ph), 7.58 (s, 1H, 4-Ox), 7.90 (s, 1H, 2-Ox), 8.04 (br, 1H, NH).

### Example 67

### Synthesis of 2-methoxy-5-(oxazol-5-yl) aniline (67)

Add 10% Pd/C (0.67 g) and anhydrous ethanol (33 ml) into 5-(3-nitro-4-methoxy)oxazole (3.3 g, 15 mmol), hydrogenate under 35-45 psi H₂, until no starting material exists, filter the reaction mixture, wash with ethanol (3×2.5 ml), concentrate the solution to 8 ml, add in hexane (12 ml), keep stand still before filtering, dry in vacuo to obtain 2.4 g product 67, yield: 84.1%.

¹H NMR (CDC₃, δ) 3.89 (s, 3H, —OCH₃), 3.98 (br, 2H, NH₂), 6.82 (d, J=10 Hz, 1H, 3-Ph), 7.02 (s, 1H, 6-Ph), 7.04 (d, J=10.5 Hz, 1H, 4-Ph), 7.19 (s, 1H, 4-Ox), 7.84 (s, 1H, 2-Ox).

### Example 68

### Synthesis of N-[3-methoxy-4-(oxazol-5-yl)-phenyl methanesulfonamide (68)

Add dichloromethane (6.5 ml), pyridine (0.2 ml) into 3-methoxy-4-(oxazol-5-yl) aniline (380.4 mg, 2 mmol) in a 25 ml flask at 2-4° C., slowly add methanesulfonyl chloride (0.2 ml) dropwise, slowly warm to room temperature after stirring for 45 minutes. Add hexane (12 ml), water (12 ml), dichloromethane (20 ml×3), filter the mixture and wash with water. Dry in vacuo to obtain 240 mg product 68 as a brown solid, yield: 44.8%.

¹H NMR (CDC₃, δ) 3.03 (s, 3H, —SO₂CH₃), 3.98 (s, 3H, —OCH₃), 6.59 (br, 1H, —NH), 6.97 (s, 1H, 2-Ph), 6.83 (d, J=10.5 Hz, 1H, 6-Ph), 7.53 (s, 1H, 4-Ox), 7.54 (d, J=10.5 Hz, 1H, 5-Ph), 7.92 (s, 1H, 2-Ox).

### Example 69

### Synthesis of N-[3-methoxy-4-(oxazol-5-yl)-phenyl toluenesulfonamide (69)

Use 3-methoxy-4-(oxazol-5-yl) aniline and p-toluenesulfonyl chloride as raw materials, follow the similar procedures applied in Example 68 to obtain compound 69.

¹H NMR (CDC₃, δ) 2.35 (s, 3H, 4′-CH₃), 3.89 (s, 3H, —OCH₃), 6.26 (s, 1H, 2-Ph), 6.39 (d, J=8.5 Hz, 1H, 6-Ph), 7.33 (s, 1H, 4-Ox), 7.34 (m, 2H, 3′, 5′-Ph), 7.55 (d, J=8.5 Hz, 1H, 5-Ph), 7.80 (m, 2H, 4′, 6′-Ph), 7.82 (s, 1H, 2-Ox).

### Example 70

### Synthesis of 3-[3-methoxy-4-(oxazol-5-yl)-anilino]propanesulfonic acid (70)

Dissolve 3-methoxy-4-(oxazol-5-yl) aniline (190.2 mg, 1 mmol) and 1,3-propane sulfonyl chloride (1.1 mmol) into methanol in a 25 ml flask, reflux for 6 h, cool and then filter out the precipitated product 70 as a yellow solid, yield: 30.5%.

¹H NMR (DMSO-d6, δ) 1.87 (in, 2H, —CH₂CH₂CH₂—), 2.56 (m, 214, —CH₂CH₂CH₂), 3.22 (m, 2H, —CH₂CH₂CH₂—), 3.87 (s, 3H, —OCH₃), 5.75 (s, 1H, —NH), 6.46 (m, 1H, 6-Ph), 6.54 (s, 1H, 2-Ph), 7.47 (d, J=10 Hz, 1H, 5-Ph), 7.27 (s, 1H, 4-Ox), 8.27 (s, 1H, 2-Ox).

### Example 71

### Synthesis of N-(3,7-dimethyl-octa-2,6-dienyl)-3-methoxy-4-(oxazol-5-yl) aniline (71)

Use 3-methoxy-4-(oxazol-5-yl) aniline and citral as the raw materials, follow the similar procedures applied in Example 1 to obtain compound 71, yield: 35.3%.

¹H NMR (CDC₃, δ) 1.61 (s, 3H, 3′-CH₃), 1.71 (s, 6H, 7′-C(CH₃)₂), 2.09 (m, 4H, 4′, 5′-CH₂CH₂—), 3.76 (m, 2H, —CH₂—), 3.89 (s, 3H, —OCH₃), 5.11 (m, 1H, 6′-H), 5.35 (1H, m, 2′-H), 6.20 (s, 1H, 2-Ph), 6.29 (d, J=8.5 Hz, 1H, 6-Ph), 7.32 (s, 1H, 4-Ox), 7.56 (d, J=8.5 Hz, 1H, 5-Ph), 7.81 (s, 1H, 2-Ox).

### Example 72

### Synthesis of N-[2-(5-methylthien-2-yl)-ethyl]-3-methoxy-4-(oxazol-5-yl) aniline (72)

Dissolve 3-methoxy-4-(oxazol-5-yl) aniline (1 mmol) into a solvent (e.g. dichloromethane), add in a small amount of pyridine or triethylamine, keep at low temperature, add in 5-methyl-(2-chloroethyl)thiophene (1 mmol), stir for 2 hours, extract with dichloromethane, wash the extractant with water, Flash separate to obtain product 72, yield: 43.2%.

¹H NMR (CDC₃, δ) 2.44 (s, 3H, CH₃—Th), 2.78 (t, 2H, —CH₂—), 3.39 (t, 2H, —NHCH₂—), 3.88 (s, 3H, —OCH₃), 6.25 (s, 1H, 2-Ph), 6.36 (d, J=8.5 Hz, 1H, 6-Ph), 6.61 (d, J=3 Hz, 1H, 4-Th), 6.82 (d, J=3 Hz, 1H, 3-Th), 7.34 (s, 1H, 4-Ox), 7.56 (d, J=8.5 Hz, 1H, 5-Ph), 7.80 (s, 1H, 2-Ox).

### Example 73

### Synthesis of N-[2-(5-bromothien-2-yl)-ethyl]-3-methoxy-4-(oxazol-5-yl) aniline (73)

Use 3-methoxy-4-(oxazol-5-yl) aniline and 2-bromo-5-(2-bromoethyl)thiophene as starting materials, follow the similar procedures applied in Example 72 to obtain compound 73.

¹H NMR (CDC₃, δ) 2.77 (t, 2H, —CH₂—), 3.39 (t, 2H, —NHCH₂—), 3.89 (s, 3H, —OCH₃), 6.24 (s, 1H, 2-Ph), 6.36 (d, J=8.5 Hz, 1H, 6-Ph), 6.67 (d, J=3 Hz, 1H, 4-Th), 6.80 (d, J=3 Hz, 1H, 3-Th), 7.34 (s, 1H, 4-Ox), 7.57 (d, J=8.5 Hz, 1H, 5-Ph), 7.81 (s, 1H, 2-Ox).

### Example 74

### Synthesis of N-(1,2-benzisoxazol-3-yl ethyl)-3-methoxy-4-(oxazol-5-yl) aniline (74)

Use 3-methoxy-4-(oxazol-5-yl) aniline and 3-(2-bromoethyl)-benzisoxazole asraw materials, follow the similar procedures applied in Example 72 to obtain compound 74.

¹H NMR (CDC₃, δ) 2.78 (t, 2H, —CH₂—), 3.39 (t, 2H, —NHCH₂—), 3.88 (s, 3H, —OCH₃), 6.25 (s, 1H, 2-Ph), 635 (d, J=8.5 Hz, 1H, 6-Ph), 6.96 (t, 1H, 5-Ben), 7.16 (t, 1H, 6-Ben), 7.29 (dd, J=8 Hz, 8 Hz, 2H, 4, 7-Ben), 7.34 (s, 1H, 4-Ox), 7.55 (d, J=8.5 Hz, 1H, 5-Ph), 7.80 (s, 1H, 2-Ox).

### Example 75

### Synthesis of N-(dibenzofuran-4-yl methyl)-3-methoxy-4-(oxazol-5-yl) aniline (75)

Use 3-methoxy-4-(oxazol-5-yl) aniline and dibenzofuran-4-carbaldehyde as starting materials, follow the similar procedures applied in Example 1 to obtain compound 75.

¹H NMR (CDC₃, δ) 4.38 (s, 2H, —CH₂), 6.48 (s, 1H, 2-Ph), 6.38 (d, J=8.5 Hz, 1H, 6-Ph), 6.99 (d, J=8 Hz, 1H, 3-Fu), 7.02 (dd, J=8 Hz, 8 Hz, 1H, 2-Fu), 7.16 (m, 2H, 7, 8-Fu), 7.31 (d, J=8 Hz, 1H, 1-Fu), 7.34 (s, 1H, 4-Ox), 7.43 (d, J=8 Hz, 1H, 6-Fu), 7.49 (d, J=8 Hz, 1H, 9-Fu), 7.58 (d, J=8.5 Hz, 1H, 5-Ph), 7.82 (s, 1H, 2-Ox).

### Example 76

### Synthesis of N-(5-methylthien-2-yl methyl)-3-methoxy-4-(oxazol-5-yl)benzylamine (76)

Use 3-methoxy-4-(oxazol-5-yl)-benzylamine and 5-methylthiophene-2-aldehyde as starting materials, follow the similar procedures applied in Example 1 to obtain compound 76, yield: 53.0%.

¹H NMR (CDC₃, δ) 2.43 (s, 3H, CH₃—Th), 3.81 (s, 2H, —NHCH₂—), 3.81 (s, 2H, —CH₂NH—), 3.88 (s, 3H, —OCH₃), 6.60 (d, J=3 Hz, 1H, 4-Th), 6.62 (s, 1H, 2-Ph), 6.76 (d, J=8.5 Hz, 1H, 6-Ph), 6.81 (d, J=3 Hz, 1H, 3-Th), 7.34 (s, 1H, 4-Ox), 7.59 (d, J=8.5 Hz, 1H, 5-Ph), 7.81 (s, 1H, 2-Ox).

### Example 77

### Synthesis of N-(5-chlorothien-2-ylmethyl)-3-methoxy-4-(oxazol-5-yl)benzylamine (77)

Use 3-methoxy-4-(oxazol-5-yl)5-chloro-benzylamine and thiophene-2-aldehyde as starting materials, follow the similar procedures applied in Example 1 to obtain compound 77.

¹H NMR (CDC₃, δ) 3.81 (s, 2H, —NHCH₂—), 3.81 (s, 2H, —CH₂NH—), 3.89 (s, 3H, —OCH₃), 6.61 (s, 1H, 2-Ph), 6.71 (d, J=3 Hz, 1H, 4-Th), 6.75 (d, J=8.5 Hz, 1H, 6-Ph), 6.81 (d, J=3 Hz, th, 3-Th), 7.35 (s, 1H, 4-Ox), 7.59 (d, J=8.5 Hz, 1H, 5-Ph), 7.82 (s, 1H, 2-Ox).

### Example 78

### Synthesis of N-(benzo[1,2,5]oxadiazol-4-yl methyl)-3-methoxy-4-(oxazol-5-yl)benzylamine (78)

Use 3-methoxy-4-(oxazol-5-yl)-benzylamine and benzo[1,2,5]oxadiazole-4-aldehyde as starting materials, follow the similar procedures applied in Example 1 to obtain compound 78.

¹H NMR (CDC₃, δ) 3.82 (s, 2H, —NHCH₂—), 3.81 (s, 2H, —CH₂NH—), 3.88 (s, 3H, —OCH₃), 6.62 (s, 1H, 2-Ph), 6.74 (d, J=8.5 Hz, 1H, 6-Ph), 7.09 (d, J=8 Hz, 1H, 5-Ben), 7.41 (dd, J=8 Hz, 8 Hz, 1H, 6-Ben), 7.34 (s, 1H, 4-Ox), 7.59 (d, J=8.5 Hz, 1H, 5-Ph), 7.73 (d, J=8 Hz, 1H, 7-Ben), 7.82 (s, 1H, 2-Ox).

### Example 79

### Synthesis of 1-(2-methyl-5-oxazol-5-yl anilino)methyl-naphthol-2 (79)

Use 2-methyl-5-(oxazol-5-yl)-phenylamine and 2-hydroxynaphthaldehyde-1 as the raw materials, follow the similar procedures applied in Example 1 to obtain compound 79.

¹H NMR (CDC₃, δ) 2.36 (s, 3H, CH3), 4.76 (s, 2H, —CH₂—), 4.03 (s, 1H, —NH), 5.02 (br, 1H, OH), 6.83 (d, J=8 Hz, 1H, 3-Naph), 6.91 (d, J=8.5 Hz, 1H, 3-Ph), 6.92 (s, 1H, 6-Ph), 7.19 (s, 1H, 4-Ox), 7.24 (m, 2H, 6, 7-Naph), 7.36 (d, J=8.5 Hz, M, 4-Ph), 7.45 (d, J=8 Hz, 1H, 4-Naph), 7.61 (dd, J=8 Hz, 8 Hz, 2H, 5, 8-Naph), 7.81 (s, 1H, 2-Ox).

### Example 80

### Synthesis of N-(2-benzothien-2-yl-ethyl) N-(3-methoxy-4-oxazol-5-yl)-2-bromo propionamide (80)

Use 3-methoxy-4-(oxazol-5-yl) aniline and 2-(2-bromoethyl)-benzothiophene as raw materials, follow the similar procedures applied in Example 72 to obtain compound N-(2-phenyl and thien-2-yl ethyl) 3-methoxy-4-(oxazol-5-yl) aniline, then follow the similar procedures applied in Example 32, with an exception of using 2-bromo-propionyl chloride as the acylating agent, to obtain compound 80.

¹H NMR (CDC₃, δ) 1.97 (d, J=8 Hz, 3H, COBrCHCH₃), 2.70 (t, 2H, —CH₂—), 3.71 (t, 2H, —NHCH₂—), 3.89 (s, 3H, —OCH₃), 4.55 (q, 1H, COBrCHCH₃), 6.65 (s, 1H, 2-Ph), 6.72 (d, J=8.5 Hz, 1H, 6-Ph), 7.24 (s, 1H, 3-Th), 7.27 (m, 1H, 5-Th), 7.32 (m, 1H, 6-Th), 7.34 (s, 1H, 4-Ox), 7.58 (d, J=8.5 Hz, 1H, 5-Ph), 7.70 (m, 1H, 4-Th), 7.77 (m, 1H, 7-Th), 7.81 (s, 1H, 2-Ox).

### Example 81

### Synthesis of N-[2-(4-chlorocinnolin-3-yl)-ethyl]-3-methoxy-4-(oxazol-5-yl) aniline (81)

Use 3-methoxy-4-(oxazol-5-yl) aniline and 4-chloro-3-(2-chloroethyl) cinnoline as raw materials, follows the similar procedures applied in Example 72 to obtain compound 81.

¹H NMR (CDC₃, δ) 2.78 (t, 2H, —CH₂—), 3.38 (t, 2H, —NHCH₂—), 3.88 (s, 3H, —OCH₃), 6.24 (s, 1H, 2-Ph), 6.36 (d, J=8.5 Hz, 1H, 6-Ph), 7.34 (s, 1H, 4-Ox), 7.58 (d, J=8.5 Hz, 1H, 5-Ph), 7.63 (dd, J=8 Hz, 8 Hz, 1H, 7-Cin), 7.73 (dd, J=8 Hz, 8 Hz, 1H, 6-Cin), 7.80 (s, 1H, 2-Ox), 8.07 (d, J=8 Hz, 1H, 5-Cin), 8.37 (d, J=8 Hz, 1H, 8-Cin).

### Example 82

### Synthesis of N-[2-(5-chlorobenzathien-2-yl)-ethyl]-3-methoxy-4-(oxazol-5-yl) aniline (82)

Use 3-methoxy-4-(oxazol-5-yl) aniline and 2-(2-bromoethyl)-5-chlorobenzothiophene as raw materials, follow the similar procedures applied in Example 72 to obtain compound 82.

¹H NMR (CDC₃, δ) 2.78 (t, 2H, —CH₂—), 3.38 (t, 2H, —NHCH₂—), 3.88 (s, 3H, —OCH₃), 6.24 (s, 1H, 2-Ph), 6.34 (d, J=8.5 Hz, 1H, 6-Ph), 7.25 (s, 1H, 3-Th), 7.33 (d, J=8 Hz, 1H, 6-Th), 7.34 (s, 1H, 4-Ox), 7.56 (d, J=8.5 Hz, 1H, 5-Ph), 7.71 (s, 1H, 4-Th), 7.72 (d, J=8 Hz, 1H, 7-Th), 7.80 (s, 1H, 2-Ox).

### Example 83

### Synthesis of N-(4,6-dichloro-2H-thiobenzopyran-3-yl methyl)-3-methoxy-4-(oxazol-5-yl) aniline (83)

Use 3-methoxy-4-(oxazol-5-yl) aniline and 4,6-dichloro-2H-thiobenzopyran-3-carbaldehyde as raw materials, follow the similar procedures applied in Example 1 to obtain compound 83, yield: 46.2%.

¹H NMR (CDC₃, δ) 3.88 (s, 3H, —OCH₃), 4.10 (dd, J=20 Hz, 20 Hz, 2H, 2-Th), 4.41 (s, 2H, —CH₂—), 7.22 (d, J=8 Hz, 1H, 7-Th), 7.27 (d, J=8 Hz, 1H, 8-Th), 7.34 (s, 1H, 4-Ox), 7.41 (s, 1H, 5-Th), 7.81 (s, 1H, 2-Ox).

### Example 84

### Synthesis of N-(3-methoxy-4-oxazole-5-yl-benzyl) N-(5-methyl-benzothien-2-ylmethyl)methylsulfonamide (84)

Use 3-methoxy-4-(oxazol-5-yl)-benzylamine and 5-methyl-benzothiophene-2-carbaldehyde as starting materials, follow the similar procedures applied in Example 1 to isolate N-(5-methyl-benzothiophene-2-methyl) 3-methoxy-4-(oxazol-5-yl)-benzylamine, add in anhydrous acetonitrile, 60% NaH, add in MsCl dropwise at 5° C., then stir at ambient temperature until the disappearance of the intermediate benzylamine, isolate and purify the product to obtain compound 84, yield: 33.2%.

¹H NMR (CDC₃, δ) 2.35 (s, 3H, CH₃—Th), 2.84 (s, 3H, SO₂CH₃), 3.81 (s, 2H, —NHCH₂—), 3.81 (s, 2H, —CH₂NH—), 3.89 (s, 3H, —OCH₃), 6.63 (s, 1H, 2-Ph), 6.75 (d, J=8.5 Hz, 1H, 6-Ph), 6.94 (s, 1H, 3-Th), 7.12 (d, J=8 Hz, 1H, 6-Th), 7.33 (s, 1H, 4-Ox), 7.55 (d, J=8.5 Hz, 1H, 5-Ph), 7.58 (s, 1H, 4-Th), 7.80 (s, 1H, 2-Ox), 7.83 (d, J=8 Hz, 1H, 7-Th).

### Example 85

### Synthesis of N-(benzothien-3-yl methyl)-3-methoxy-4-(oxazol-5-yl)benzylamine (85)

Use 3-methoxy-4-(oxazol-5-yl)-benzylamine and benzothiophene-3-carbaldehyde as starting materials, follow the similar procedures applied in Example 1 to obtain compound 85.

¹H NMR (CDC₃, δ) 3.81 (s, 2H, —NHCH₂—), 3.82 (s, 2H, —CH₂NH—), 3.86 (s, 3H, —OCH₃), 6.62 (s, 1H, 2-Ph), 6.74 (d, J=8.5 Hz, 1H, 6-Ph), 7.33 (s, 1H, 4-Ox), 7.38 (s, 1H, 2-Th), 7.41 (m, 2H, 5, 6-Th), 7.54 (d, J=8.5 Hz, 1H, 5-Ph), 7.81 (s, 1H, 2-Ox), 7.83 (d, J=8 Hz, 1H, 4-Th), 7.86 (d, J=8 Hz, 1H, 7-Th).

### Example 86

### Synthesis of N-(1-methyl-1H-indazole-5-methyl)-3-methoxy-4-(oxazol-5-yl)benzylamine (86)

Use 3-methoxy-4-(oxazol-5-yl)-benzylamine and 5-(bromomethyl)-1-methyl-1H-indazole as raw material, follow the similar procedures applied in Example 72 to obtain compound 86.

¹H NMR (CDC₃, δ) 3.80 (s, H, CH₃—Ind), 3.81 (s, 2H, —NHCH₂—), 3.81 (s, 2H, —CH₂NH—), 3.88 (s, 3H, —OCH₃), 6.65 (s, 1H, 2-Ph), 6.74 (d, J=8.5 Hz, 1H, 6-Ph), 7.15 (d, J=8 Hz, 1H, 6-Ind), 7.34 (s, 1H, 4-Ox), 7.49 (d, J=8 Hz, 1H, 7-Ind), 7.57 (d, J=8.5 Hz, 1H, 5-Ph), 7.66 (s, 1H, 4-Ind), 7.81 (s, 1H, 2-Ox), 8.14 (s, 1H, 3-Ind).

### Example 87

### Synthesis of N-methyl-iv-(5-chlorobenzothien-3-yl methyl)-3-methoxy-4-(oxazol-5-yl)benzylamine (87)

Use 3-methoxy-4-(oxazol-5-yl)-benzylamine and 5-chlorobenzothiophene-3-carbaldehyde as starting materials, follow the similar procedures applied in Example 12 to obtain compound 87.

¹H NMR (CDC₃, δ) 2.27 (s, 3H, N—CH₃), 3.62 (s, 2H, —NHCH₂—), 3.62 (s, 2H, —CH₂NH—), 3.88 (s, 3H, —OCH₃), 6.64 (s, 1H, 2-Ph), 6.75 (d, J=8.5 Hz, 1H, 6-Ph), 7.34 (s, 1H, 4-Ox), 7.37 (s, 1H, 2-Th), 7.40 (d, J=8 Hz, 1H, 6-Th), 7.58 (d, J=8.5 Hz, 1H, 5-Ph), 7.82 (s, 1H, 2-Ox), 7.83 (s, 1H, 4-Th), 7.84 (d, J=8 Hz, 1H, 7-Th).

### Example 88

### Synthesis of N-(10-methyl-10H-phenothiazin-3-yl methyl)-3-methoxy-4-oxazol-5-yl aniline (88)

Use 3-methoxy-4-(oxazol-5-yl) aniline and 10-methyl-10H-phenothiazine carbaldehyde-3 as raw materials, follow the similar procedures applied in Example 1 to obtain compound 88, yield: 41.5%.

¹H NMR (CDC₃, δ) 2.79 (s, 3H, NCH₃-Phe), 3.89 (s, 3H, —OCH₃), 4.42 (s, 2H, —CH₂—), 6.28 (s, 1H, 2-Ph), 6.34 (d, J=8.5 Hz, 1H, 6-Ph), 6.56 (d, J=8 Hz, 1H, 5-Phe), 6.69 (d, J=8 Hz, 1FI, 9-Phe), 6.72 (s, 1H, 2-Phe), 6.76 (dd, J=8 Hz, 8 Hz, 1H, 7-Phe), 6.79 (d, J=8 Hz, 1H, 4-Phe), 6.92 (d, J=8 Hz, 1H, 6-Phe), 6.99 (dd, J=8 Hz, 8 Hz, 1H, 8-Phe), 7.34 (s, 1H, 4-Ox), 7.57 (d, J=8.5 Hz, 1H, 5-Ph), 7.81 (s, 1H, 2-Ox).

### Example 89

### Synthesis of N-(5-methylthien-2-yl methyl)-3-chloride-4-oxazol-5-yl benzylamine (89)

Use 3-chloro-4-(oxazol-5-yl)benzylamine and 5-methylthiophene-2-carbaldehyde as starting materials, follow the similar procedures applied in Example 1 to obtain compound 89.

¹H NMR (CDC₃, δ) 2.43 (s, 3H, CH₃—Th), 3.81 (s, 2H, —NHCH₂—), 3.82 (s, 2H, —CH₂NH—), 6.61 (d, J=3 Hz, 1H, 4-Th), 6.82 (d, J=3 Hz, 1H, 3-Th), 7.06 (d, J=8.5 Hz, 1H, 6-Ph), 7.12 (s, 1H, 2-Ph), 7.33 (s, 1H, 4-Ox), 7.61 (d, J=8.5 Hz, 1H, 5-Ph), 7.82 (s, 1H, 2-Ox).

### Example 90

### Synthesis of N-dibenzothien-4-ylmethyl-3-chloro-4-oxazol-5-yl aniline (90)

Use 3-chloro-4-(oxazol-5-yl) aniline, and dibenzo[b,d]thiophene-4-carbaldehyde as starting materials, follow the similar procedures applied in Example 1 to obtain compound 90, yield: 48.1%.

¹H NMR (CDC₃, δ) 4.31 (s, 2H, —CH₂), 6.49 (s, 1H, 2-Ph), 6.37 (d, J=8.5 Hz, 1H, 6-Ph), 7.12 (d, J=8 Hz, 1H, 3-Th), 7.22 (dd, J=8 Hz, 8 Hz, 1H, 2-Th), 730 (m, 2H, 7, 8-Th), 7.33 (s, 1H, 4-Ox), 7.57 (d, J=8.5 Hz, 1H, 5-Ph), 7.59 (d, J=8 Hz, 1H, 1-Th), 7.77 (d, J=8 Hz, 1H, 9-′Fh), 7.81 (s, 1H, 2-Ox), 7.87 (d, J=8 Hz, 1H, 6-Th).

### Example 91

### Synthesis of N-(benzo[1,2,5]-thiadiazol-4-yl methyl)-3-chloro-4-oxazol-5-yl benzylamine (91)

Use 3-chloro-4-(oxazol-5-yl)-benzylamine and 2,1,3-benzothiadiazole-4-carbaldehyde as a starting material, follow the similar procedures applied in Example 1 to obtain compound 91.

¹H NMR (CDC₃, δ) 3.81 (s, 2H, —NHCH₂—), 3.81 (s, 2H, —CH₂NH—), 7.02 (d, J=8.5 Hz, 1H, 6-Ph), 7.10 (s, 1H, 2-Ph), 7.32 (d, J=8 Hz, 1H, 5-Ben), 7.41 (dd, J=8 Hz, 8 Hz, 1H, 6-Ben), 7.34 (s, 1H, 4-Ox), 7.58 (d, J=8.5 Hz, 1H, 5-Ph), 7.78 (d, J=8 Hz, 1H, 7-Ben), 7.81 (s, 1H, 2-Ox).

### Example 92

### Synthesis of 3-(3-methoxy-4-oxazol-5-yl anilino)methyl 4-H-benzopyranol-6 (92)

Use 3-methoxy-4-(oxazol-5-yl) aniline and 6-hydroxybenzopyran-3-carbaldehyde as starting materials, follow the similar procedures applied in Example 1 to obtain compound 92, yield: 38.6%.

¹H NMR (CDC₃, δ) 3.22 (m, 2H, 3-Chro), 3.88 (s, 3H, —OCH₃), 6.22 (s, 1H, 2-Chro), 6.27 (s, 1H, 2-Ph), 6.36 (s, 1H, 5-Chro), 6.36 (d, J=8.5 Hz, 1H, 6-Ph), 6.37 (d, J=12 Hz, 7-Chro), 6.45 (d, J=12 Hz, 8-Chro), 7.34 (s, 1H, 4-Ox), 7.57 (d, J=8.5 Hz, 1H, 5-Ph), 7.81 (s, 1H, 2-Ox).

### Example 93

### Synthesis of N-(5-bromofuran-2-yl methyl)-3-methoxy-4-(oxazol-5-yl)benzylamine (93)

Use 3-methoxy-4-(oxazol-5-yl)-benzylamine and 5-bromo-furaldehyde-2 as starting materials, follow the similar procedures applied in Example 1 to obtain compound 93.

¹H NMR (CDC₃, δ) 3.66 (s, 2H, —NHCH₂—), 3.81 (s, 2H, —CH₂NH—), 3.89 (s, 3H, —OCH₃), 6.13 (d, J=3 Hz, 1H, 3-Fu), 6.20 (d, J=3 Hz, 1H, 4-Fu), 6.63 (s, 1H, 2-Ph), 6.74 (d, J=8.5 Hz, 1H, 6-Ph), 7.34 (s, 1H, 4-Ox), 7.58 (d, J=8.5 Hz, 1H, 5-Ph), 7.81 (s, 1H, 2-Ox).

### Example 94

### Synthesis of N-(2-methylacridine-9-methyl)-3-methoxy-4-(oxazol-5-yl) aniline (94)

Use 3-methoxy-4-(oxazol-5-yl) aniline and 2-methyl-9-acridine carbaldehyde as raw materials, follow the similar procedures applied in Example 1 to obtain compound 94.

¹H NMR (CDC₃, δ) 2.36 (s, 3H, CH₃-Acr), 3.88 (s, 3H, —OCH₃), 4.28 (s, 2H, —CH₂₇), 6.28 (s, 1H, 2-Ph), 6.34 (d, J=8.5 Hz, 1H, 6-Ph), 7.32 (s, 1H, 4-Ox), 7.41 (m, 1H, 7-Acr), 7.46 (d, J=8 Hz, 1H, 3-Acr), 7.47 (s, 1H, 1-Acr), 7.56 (d, J=8.5 Hz, 1H, 5-Ph), 7.59 (m, 1H, 6-Acr), 7.78 (d, J=8 Hz, 1H, 8-Acr), 7.81 (s, 1H, 2-Ox) 7.99 (d, J=8 Hz, 1H, 4-Acr), 8.03 (d, J=8 Hz, 1H, 5-Acr).

### Example 95

### Synthesis of N-(anthracen-9-yl methyl)-3-methoxy-4-(oxazol-5-yl) aniline (95)

Use 3-methoxy-4-(oxazol-5-yl)-benzylamine and 9-anthracene carbaldehyde as the starting materials, follow the similar procedures applied in Example 1 to obtain compound 95.

¹H NMR (CDC₃, δ) 3.87 (s, 3H, —OCH₃), 4.76 (s, 2H, —CH₂—), 6.29 (s, 1H, 2-Ph), 6.35 (d, J=8.5 Hz, 1H, 6-Ph), 7.31 (s, 1H, 4-Ox), 7.38 (m, 4H, 2, 3, 6, 7-Anth), 7.56 (d, J=8.5 Hz, 1H, 5-Ph), 7.81 (s, 1H, 2-Ox), 7.89 (dd, J=8 Hz, 8 Hz, 2H, 4, 5-Anth), 7.97 (dd, J=8 Hz, 8 Hz, 2H, 1, 8-Anth), 8.15 (s, 1H, 10-Anth).

### Example 96

### Synthesis of 5-(3-methoxy-4-oxazole-5-yl anilinomethyl furan-2-yl methanol (96)

Use 3-methoxy-4-(oxazol-5-yl) aniline and 5-hydroxyfuraldehyde-2 as starting materials, follow the similar procedures applied in Example 1 to obtain compound 96.

¹H NMR (CDC₃, δ) 3.90 (s, 3H, —OCH₃), 4.30 (s, 2H, —CH₂), 4.63 (s, 2H, —CH₂OH) 5.97 (d, J=2.5 Hz, 1H, 3-Fu), 6.16 (d, J=2.5 Hz, 1H, 3-Fu), 6.27 (s, 1H, 2-Ph), 6.35 (d, J=8.5 Hz, 1H, 6-Ph), 7.34 (s, 1H, 4-Ox), 7.56 (d, J=8.5 Hz, 1H, 5-Ph), 7.80 (s, 1H, 2-Ox).

### Example 97

### Synthesis of N-(4-bromo-5-ethylthien-2-yl methyl)-3-methoxy-4-(oxazol-5-yl) aniline (97)

Use 3-methoxy-4-(oxazol-5-yl) aniline and 4-bromo-5-ethyl-thiophene-2-carbaldehyde as starting materials, follow the similar procedures applied in Example 1 to obtain compound 97, yield 56.2%.

¹H NMR (CDC₃, δ) 1.28 (t, 3H, —CH₂CH₃), 2.81 (q, 2H, —CH₂CH₃), 3.88 (s, 3H, —OCH₃), 4.48 (s, 2H, —CH₂—), 6.25 (s, 1H, 2-Ph), 6.35 (d, J=8.5 Hz, 1H, 6-Ph), 6.76 (s, 1H, 3-Th), 7.34 (s, 1H, 4-Ox), 7.55 (d, J=8.5 Hz, 1H, 5-Ph), 7.81 (s, 1H, 2-Ox).

### Example 98

### Synthesis of N-(thien-2-yl sulfonyl)-3-methoxy-4-oxazole-5-yl aniline (98)

Use 3-methoxy-4-(oxazol-5-yl) aniline and thiophene-2-sulfonyl chloride as starting materials, follow the similar procedures applied in Example 68 to obtain compound 98, yield: 53.3%.

¹H NMR (CDC₃, δ) 3.88 (s, 3H, —OCH₃), 6.25 (s, 1H, 2-Ph), 6.39 (d, J=8.5 Hz, 1H, 6-Ph), 7.00 (m, 1H, 3-Th), 7.04 (m, 1H, 4-Th), 7.24 (m, 1H, 5-Th), 7.34 (s, 1H, 4-Ox), 7.53 (d, J=8.5 Hz, 1H, 5-Ph), 7.82 (s, 1H, 2-Ox).

### Example 99

### Synthesis of N-(5-chloro-4-nitro-thien-2-yl sulfonyl)-3-methoxy-4-oxazole-5-yl aniline (99)

Use 3-methoxy-4-(oxazol-5-yl) aniline and 5-chloro-4-nitro-thiophene-2-yl sulfonyl chloride as starting materials, follow the similar procedures applied in Example 1 to obtain compound 99, yield: 49.3%.

¹H NMR (CDC₃, δ) 3.88 (s, 3H, —OCH₃), 6.24 (s, 1H, 2-Ph), 6.38 (d, J=8.5 Hz, 1H, 6-Ph), 6.81 (s, 1H, 3-Th), 7.34 (s, 1H, 4-Ox), 7.55 (d, J=8.5 Hz, 1H, 5-Ph), 7.82 (s, 1H, 2-Ox).

### Example 100

### Synthesis of N-(2,3-dihydrobenzofuro-5-yl methyl)-3-methoxy-4-(oxazol-5-yl) aniline (100)

Use 3-methoxy-4-(oxazol-5-yl) aniline and 2,3-dihydro-benzofuraldehyde-5 as starting materials, follow the similar procedures applied in Example 1 to obtain compound 100, yield: 68.3%.

¹H NMR (CDC₃, δ) 2.96 (m, 2H, 3-Fu), 3.89 (s, 3H, —OCH₃), 4.27 (m, 2H, 2-Fu), 4.49 (s, 2H, —CH₂—), 6.25 (s, 1H, 2-Ph), 6.38 (d, J=8.5 Hz, 1H, 6-Ph), 6.60 (d, J=8 Hz, 1H, 7-Fu), 6.78 (d, J=8 Hz, 1H, 6-Fu), 6.81 (s, 1H, 4-Fu), 7.33 (s, 1H, 4-Ox), 7.57 (d, J=8.5 Hz, 1H, 5-Ph), 7.81 (s, 1H, 2-Ox).

### Example 101

### Synthesis of N-(5-acetyl thien-2-yl methyl)-3-methoxy-4-oxazole-5-yl aniline (101)

Use 3-methoxy-4-(oxazol-5-yl) aniline and 5-acetyl-thiophene-2-carbaldehyde as starting materials, follow the similar procedures applied in Example 1 to obtain compound 101, yield: 55.3%.

¹H NMR (CDC₃, δ) 2.54 (s, 3H, —COCH₃), 3.88 (s, 3H, —OCH₃), 4.47 (s, 2H, —CH₂—), 6.25 (s, 1H, 2-Ph), 6.36 (d, J=8.5 Hz, 1H, 6-Ph), 6.64 (d, J=4 Hz, 1H, 3-Th), 7.28 (d, J=4 Hz, 1H, 4-Th), 7.34 (s, 1H, 4-Ox), 7.58 (d, J=8.5 Hz, 1H, 5-Ph), 7.82 (s, 1H, 2-Ox).

### Example 102

### Synthesis of N-(5-methylthiothien-2-yl methyl)-3-methoxy-4-(oxazol-5-yl) aniline (102)

Use 3-methoxy-4-(oxazol-5-yl) aniline and 5-methylmercaptothiophene-2-carbaldehyde as starting materials, follow the similar procedures applied in Example 1 to obtain compound 102, yield: 41.6%.

¹H NMR (CDC₃, δ) 2.47 (s, 3H, —SCH3), 3.89 (s, 3H, —OCH₃), 4.48 (s, 2H, —CH₂—), 6.26 (s, 1H, 2-Ph), 6.35 (d, J=8.5 Hz, 1H, 6-Ph), 6.42 (d, J=4 Hz, 1H, 3-Th), 6.69 (d, J=4 Hz, 1H, 4-Th), 7.34 (s, 1H, 4-Ox), 7.55 (d, J=8.5 Hz, 1H, 5-Ph), 7.80 (s, 1H, 2-Ox).

### Example 103

### Synthesis of N-[5-(4-hydroxypiperidinyl)thien-2-yl]3-methoxy-4-oxazole-5-yl aniline (103)

Use 3-methoxy-4-(oxazol-5-yl) aniline and 5-(4-hydroxypiperidine-1-yl)-thiophene-2-carbaldehyde as starting materials, follow the similar procedures applied in Example 1 to obtain compound 103, yield 33.8%.

¹H NMR (CDC₃, δ) 1.95 (m, 4H, 3, 5-Pyd), 3.38 (m, 4H, 2, 6-Pyd), 3.89 (s, 3H, —OCH₃), 3.95 (m, 1H, 4-Pyd), 4.46 (s, 2H, —CH₂—), 6.26 (s, 1H, 2-Ph), 6.35 (d, J=8.5 Hz, 1H, 6-Ph), 6.15 (d, J=4 Hz, 1H, 3-Th), 5.78 (d, J=4 Hz, 1H, 4-Th), 7.34 (s, 1H, 4-Ox), 7.57 (d, J=8.5 Hz, 1H, 5-Ph), 7.81 (s, 1H, 2-Ox).

### Example 104

### Synthesis of N-[5-(2-chloro-4-trifluoromethylphenyl) furan-2-yl methyl]3-methoxy-4-oxazol-5-yl aniline (104)

Use 3-methoxy-4-(oxazol-5-yl) aniline and 5-(2-chloro-4-trifluoromethyl-phenyl)-furaldehyde-2 as starting materials, follow the similar procedures applied in Example 1 to obtain compound 104, yield: 48.8%.

¹H NMR (CDC₃, δ) 3.88 (s, 3H, —OCH₃), 4.29 (s, 2H, —CH₂—), 6.25 (s, 1H, 2-Ph), 6.36 (d, J=8.5 Hz, 1H, 6-Ph), 6.13 (d, J=4 Hz, 1H, 3-Fu), 6.20 (d, J=4 Hz, 1H, 4-Fu), 7.33 (s, 1H, 4-Ox), 7.35 (d, J=8 Hz, 1H, 6′-Ph), 7.39 (d, J=8 Hz, 1H, 5′-Ph), 7.52 (s, 1H, 3′-Ph), 7.56 (d, J=8.5 Hz, 1H, 5-Ph), 7.81 (s, 1H, 2-Ox).

### Example 105

### Synthesis of N-(furan-3-yl propen-1-yl)-3-methoxy-4-oxazol-5-yl aniline (105)

Use 3-methoxy-4-(oxazol-5-yl) aniline and 3-furan-3-yl-propionaldehyde as raw materials, follow the similar procedures applied in Example 1 to obtain compound 105, yield: 28.3%.

¹H NMR (CDC₃, δ) 3.91 (s, 3H, —OCH₃), 4.00 (d, j=5 Hz, 1H, —CH₂—), 6.30 (s, 1H, 2-Ph), 6.33 (m, 1H, —CH₂—CH═CH—), 6.36 (d, j=4 Hz, 1H, 4-Fu), 6.38 (d, j=8.5 Hz, 1H, 6-Ph), 6.64 (m, 1H, —CH₂—CH═CH—), 7.34 (s, 1H, 4-Ox), 7.39 (d, j=4 Hz, 5-Fu), 7.50 (s, 1H, 2-Fu), 7.57 (d, j=8.5 Hz, 1H, 5-Ph), 7.81 (s, 1H, 2-Ox).

### Example 106

### Synthesis of N-(benzothien-5-yl methyl)-3-methoxy-4-oxazol-5-yl aniline (106)

Use 3-methoxy-4-(oxazol-5-yl) aniline and benzothiophene-5-carbaldehyde as raw materials, follow the similar procedures applied in Example 1 to obtain compound 105, yield: 56.8%.

¹H NMR (CDC₃, δ) 3.88 (s, 3H, —OCH₃), 4.49 (s, 2H, —CH₂—), 6.25 (s, 1H, 2-Ph), 6.35 (d, J=8.5 Hz, 1H, 6-Ph), 7.11 (d, J=8 Hz, 1H, 6-Th), 7.29 (d, J=4 Hz, 1H, 3-Th), 7.34 (s, 1H, 4-Ox), 7.40 (d, J=4 Hz, 1H, 2-Th), 7.57 (d, J=8.5 Hz, 1H, 5-Ph), 7.58 (s, 1H, 4-Th), 7.74 (d, J=8 Hz, 1H, 7-Th), 7.81 (s, 1H, 2-Ox).

### Example 107

### Measurement of IMPDH Inhibitor Activity

In 96-well plates, use IMP as the substrate and NAD, NADH as chromogenic agents to determine the activity of the compounds described in this invention as IMPDH inhibitor. Add 2 μl of each of the tested sample solution, 20 μl enzyme extract and 30 μl IMP into a sample well in the 96-well plates; 2 μl DMSO and 30 μl IMP solution into a control well; 2 μl DMSO and 20 μl IMPDH buffer instead of the enzyme extract and 30 μl IMP solution into a blank well. Keep plates at constant 37° C. for 15 min, measure absorbance values A₃₄₀ (OD₁) of the solutions in various wells; subsequently, add into each well with 50 μl NAD, keep at constant 37° C. for 50 min, then measure A₃₄₀ (OD₂) values. Calculate IMPDH inhibition rates of samples as follows: Inhibition (%)=[(OD₂−OD₁)_{control}−(OD₂−OD₁)_{Sample}]/[(OD₂−OD₁)_{control}−(OD₂−OD₁)_{Blank}]×100%.

The results are shown in Table 2.

### Example 108

### Screening of Anti-B3, B6 Type Coxsackie Viruses (COX-B3, COX-B6) Activity of the Samples

Prepare stock solutions of COX-B3 (or COX-B6) virus strains as well as tested compounds in DMSO before use. Before tests, dilute the stock solution with culture solution into a certain concentration and then dilute 3 times to make a total of eight dilutions. Use ribavirin (RBV) as positive control. Test procedures are as follows: seed Vero cells into 96-well culture plates, infect with 10⁻⁵ Coxsackie virus of B3 (or B6) type, 24 hours later; adsorb subsequently for 2 hours, then discard the virus solution, add in the sample compounds and the positive control according to the abovementioned dilutions, set cell control wells and virus control wells simultaneously. When the cytopathic effect (CPE) of cells in the virus control wells reach 4+, observe CPE of the cells in each group. Use Reed-Muench method to calculate 50% inhibitory concentration (IC₅₀) of the sample compounds against coxsackie virus B3 (or B6) type. Screening results are shown in Tables 3 and 4

### Example 109

### In Vitro Human Hepatoma Cell HepG2 Inhibitory Activity Measurement

Collect well growing tumor cells, prepare 6×10⁴/ml cell suspension in RPMI-1640 or DMEM culture medium containing 10% FBS, seed in 96-well plates, 100 μl suspension each well, incubate at 37° C. under 5% CO₂ atmosphere for 24 h, add the test solution (final concentration of drug: 0.625, 1.25, 2.5, 5.0, 10 μg/ml), for each concentration set three parallel holes, set positive control (cisplatin 10, 1, 0.1 μg/ml) and a blank control simultaneously. After culture for 48 h discard the supernatants, add into each well 10 μl MTT solution (5 mg/ml, prepared with RPMI-1640 medium), continue culturing for 4 It, add into each hole 100 μl acidified isopropanol cell lysate, culture overnight, measure absorbance (A) values with Bio-Tek MQX200 type microplate reader with the detection wavelength at 540 nm, and reference wavelength 450 nm. Calculate the inhibition rate as follows: inhibition rate=(A_{blank}−Aₛₐₘₚₗₑ)/A_{blank control}×100%. Use Graphpad Prism 5 statistical software to calculate IC₅₀. Screening results are shown in Table 5.

### Example 110

### In Vitro Human Lung Adenocarcinoma Cell A549 Inhibitory Activity Measurement

Cell lines: human lung adenocarcinoma cell A549. Collect well growing tumor cells, prepare cell suspension with Ham's F12 culture solution containing 10% fetal calf serum medium, seed the suspension in 96-well plates, each well 100 μl suspension at 5000 cells/well, incubate at 37° C., under 5% CO₂ atmosphere, 24 h later, add 2×drug solution 1000, each concentration in three parallel holes, set the blank control simultaneously. After culture for 48 h, add 20 μl (5 mg/ml) MTT to each well, culture 4 h, discard the supernatants. Measure absorbance (A) values using Bio-Rad 680 microplate reader at a detection wavelength of 570 nm, the survival rate (%) is calculated as follows: AₛₐₘₚₗₑA_{blank control}×100%. Use statistical software Graphpad Prism5 to calculate IC₅₀. After primary screening of all samples, adjust the concentration gradient of the samples with IC₅₀<100 μg/ml to do additional screenings, which are carried out with two parallel determinations. Screening results are shown in Table 5.

1-10. (canceled)

11. A group of phenyl-oxazolyl derivatives or pharmaceutically acceptable salts thereof, having a general structure shown in (I): 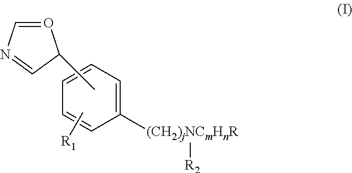
wherein:
R₁ is selected from the group consisting of H, a halogen, hydroxyl, C₁-C₃ alkyl, and C₁-C₃ alkoxyl;
R₂ is optional and selected from the group consisting of H, a substituted or unsubstituted, saturated or unsaturated C₁-C₁₂ alkyl, a carbonyl, and a sulfonyl;
R is selected from the group consisting of H, cyano group, substituted or unsubstituted, saturated or unsaturated C₁-C₁₂ alkyl, C₁-C₁₂ alkoxyl or aryloxyl, alkylmercapto or arylmercapto group, amino group, substituted amino group, sulfonic acid group, sulfonyl, substituted or unsubstituted monocyclic to tricyclic aryl group, and substituted or unsubstituted heterocyclic group;
said heterocyclic group is selected from the group consisting of a five to six membered monocyclic heterocyclic group, five to six membered bicyclic or tricyclic heterocyclic group;
wherein the heterocyclic group contains 1 to 3 hetero atoms;
wherein the heteroatoms are N, O, S;
wherein j=0-1, m=0-3, n=0-6, and j, m and n may be the same or different.

12. The group of phenyl-oxazolyl derivatives or pharmaceutically acceptable salts thereof according to claim 11, wherein the five membered monocyclic heterocyclic group is selected from the group consisting of substituted or unsubstituted thienyl, furyl, pyrrolyl, isoxazolyl, thiazolyl, imidazolyl, pyrazolyl and triazolyl.

13. The group of phenyl-oxazolyl derivatives or pharmaceutically acceptable salts thereof according to claim 11, wherein the six membered monocyclic heterocyclic group is selected from the group consisting of substituted or unsubstituted piperidinyl, pyridinyl, pyranyl, pyridazinyl, pyrimidinyl and pyrazinyl.

14. The group of phenyl-oxazolyl derivatives or pharmaceutically acceptable salts thereof according to claim 11, wherein the bicyclic heterocyclic group is selected from the group consisting of substituted or unsubstituted indolyl, benzothienyl, benzothiazolyl, benzoxazolyl, benzopyranyl, thiobenzopyranyl, quinolinyl, cinnolinyl, indazolyl, benzooxadiazolyl and benzothiadiazolyl.

15. The group of phenyl-oxazolyl derivatives or pharmaceutically acceptable salts thereof according to claim 11, wherein the tricyclic heterocyclic group is selected from the group consisting of substituted or unsubstituted dibenzofuranyl, dibenzothienyl, acridinyl, and phenothiazinyl.

16. The group of phenyl-oxazolyl derivatives or pharmaceutically acceptable salts thereof according to claim 11, wherein the group includes salts of the compound of structure (I) with an acid, wherein the acid is selected from the group consisting of mineral acids, inorganic acids, and organic acids.

17. The group of pharmaceutically acceptable salts of claim 16 wherein the acid is an inorganic acid selected from the group consisting of hydrochloric acid, hydrobromic acid, and sulfuric acid.

18. The group of pharmaceutically acceptable salts of claim 16 wherein the acid is an organic acid selected from the group consisting of acetic acid, trifluoroacetic acid, lactic acid, succinic acid, fumaric acid, maleic acid, citric acid, benzoic acid, methanesulfonic acid, and p-toluenesulfonic acid.

19. A method to prepare compounds having a general structure shown in (I), the method comprising:
providing compound A;
providing compound B;
reacting compound A and compound B as follows: 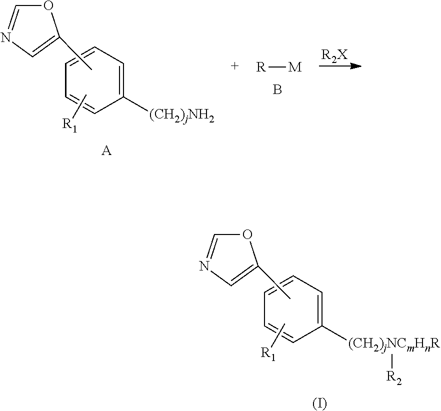
wherein:
R₁ is selected from the group consisting of H, a halogen, hydroxyl, C₁-C₃ alkyl, C₁-C₃ alkoxyl;
R₂ is optional and selected from the group consisting of H, a substituted or unsubstituted, saturated or unsaturated C₁-C₁₂ alkyl, a carbonyl, and a sulfonyl;
R is selected from the group consisting of H, cyano group, substituted or unsubstituted, saturated or unsaturated C₁-C₁₂ alkyl, C₁-C₁₂ alkoxyl or aryloxyl, alkylmercapto or arylmercapto group, amino group, substituted amino group, sulfonic acid group, sulfonyl, substituted or unsubstituted monocyclic to tricyclic aryl group, and substituted or unsubstituted heterocyclic group;
said heterocyclic group is selected from the group consisting of a five to six membered monocyclic heterocyclic group, five to six membered bicyclic or tricyclic heterocyclic group;
wherein the heterocyclic group contains 1 to 3 hetero atoms;
wherein the heteroatoms are N, O, S;
wherein j=0-1, m=0-3, n=0-6, and j, m and n may be the same or different;
wherein each of M, X is selected from the group consisting of formyl, halogen, and acyl.

20. The method of claim 19 wherein R₂ is present, the method further comprising:
mixing and dissolving compound A and compound B in a solvent, to thereby form a reactant;
reacting the reactant with R₂X to thereby produce the compounds of structure (I).

21. A pharmaceutical composition comprising a therapeutically effective amount of phenyl-oxazolyl derivatives or pharmaceutically acceptable salts thereof, having a general structure shown in (I): 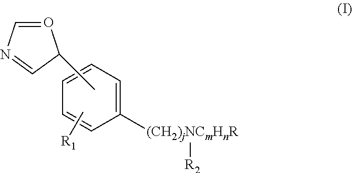
wherein:
R₁ is selected from the group consisting of H, a halogen, hydroxyl, C₁-C₃ alkyl, and C₁-C₃ alkoxyl;
R₂ is optional and selected from the group consisting of H, a substituted or unsubstituted, saturated or unsaturated C₁-C₁₂ alkyl, a carbonyl, and a sulfonyl;
R is selected from the group consisting of H, cyano group, substituted or unsubstituted, saturated or unsaturated C₁-C₁₂ alkyl, C₁-C₁₂ alkoxyl or aryloxyl, C₁-C₁₂ alkylmercapto or arylmercapto group, amino group, substituted amino group, sulfonic acid group, sulfonyl, substituted or unsubstituted monocyclic to tricyclic aryl group, and substituted or unsubstituted heterocyclic group;
said heterocyclic group is selected from the group consisting of a five to six membered monocyclic heterocyclic group, five to six membered bicyclic or tricyclic heterocyclic group;
wherein the heterocyclic group contains 1 to 3 hetero atoms;
wherein the heteroatoms are N, O, S;
wherein j=0-1, m=0-3, n=0-6, and j, m and n may be the same or different;
wherein the phenyl-oxazolyl derivatives or salts thereof are an active ingredient, and the composition also comprises at least one pharmaceutically acceptable carrier material.

22. A method of inhibiting IMPDH, the method comprising:
providing phenyl-oxazolyl derivatives or pharmaceutically acceptable salts thereof, having a general structure shown in (I): 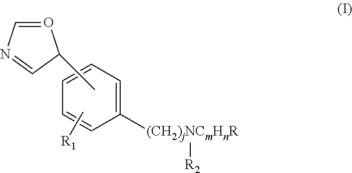
wherein:
R₁ is selected from the group consisting of H, a halogen, hydroxyl, C₁-C₃ alkyl, C₁-C₃ alkoxyl;
R₂ is optional and selected from the group consisting of H, a substituted or unsubstituted, saturated or unsaturated C₁-C₁₂ alkyl, a carbonyl, and a sulfonyl;
R is selected from the group consisting of H, cyano group, substituted or unsubstituted, saturated or unsaturated C₁-C₁₂ alkyl, C₁-C₁₂ alkoxyl or aryloxyl, C₁-C₁₂ alkylmercapto or arylmercapto group, amino group, substituted amino group, sulfonic acid group, sulfonyl, substituted or unsubstituted monocyclic to tricyclic aryl group, and substituted or unsubstituted heterocyclic group;
said heterocyclic group is selected from the group consisting of a five to six membered monocyclic heterocyclic group, five to six membered bicyclic or tricyclic heterocyclic group;
wherein the heterocyclic group contains 1 to 3 hetero atoms;
wherein the heteroatoms are N, O, S;
wherein j=0-1, m=0-3, n=0-6, and j, m and n may be the same or different, the method comprising:
administering the phenyl-oxazolyl derivatives or salts thereof of structure (I) to an organism.

23. The method of claim 22 wherein the organism is a human.

24. The method of claim 22 wherein the method is used in an antiviral therapy.

25. The method of claim 22 wherein the method is used in an anticancer therapy.

26. The method of claim 22 wherein the method is used in an immunosuppressive therapy.
